(19) United States
(12) Patent Application Publication
(10) Pub. No.: US 2024/0352068 A1
(43) Pub. Date: Oct. 24, 2024

(54) VIMENTIN TARGETED PEPTOIDS FOR EARLY DIAGNOSIS AND TREATMENT OF CANCER

(71) Applicant: UNIVERSITY OF HOUSTON SYSTEM, Houston, TX (US)

(72) Inventors: Damith Gomika UDUGAMASOORIYA, Katy, TX (US); Satya Prakash SHUKLA, Houston, TX (US); Haowen ZHANG, Houston, TX (US)

(73) Assignee: UNIVERSITY OF HOUSTON SYSTEM, Houston, TX (US)

(21) Appl. No.: 18/294,498

(22) PCT Filed: Aug. 1, 2022
(86) PCT No.: PCT/US2022/039079

§ 371 Date: Feb. 1, 2024

Related U.S. Application Data
(60) Provisional application No. 63/229,227, filed on Aug. 4, 2021.

(51) Int. Cl. C07K 7/06; A61K 38/00; A61P 35/00; G01N 33/574

(52) U.S. Cl. CPC C07K 7/06; A61P 35/00; G01N 33/57484; A61K 38/00

(57) ABSTRACT

Embodiments of the present disclosure pertains to one or more novel peptoids, derivatives thereof, multimers thereof, and combinations thereof. The peptoids may be suitable for use in treating or preventing a cancer in a subject, detecting a cancer in a subject, or as research and development tools. Further embodiments pertain to methods of inhibiting the growth of cancer cells by exposing the cancer cells to the peptoids. Additional embodiments of the present disclosure pertain to methods of treating or preventing a cancer in a subject by administering the peptoids to the subject. Further embodiments pertain to methods of detecting cancer in a subject by exposing cells susceptible of being cancerous to the peptoids, detecting the presence or absence of vimentin associated with cells susceptible of being cancerous, and correlating the presence or absence of the vimentin to the presence or absence of the cancer in the subject.

### CROSS-REFERENCE TO RELATED APPLICATIONS

The present application claims the benefit of U.S. Provisional Patent Application No. 63/229,227, filed on Aug. 4, 2021. The entirety of the aforementioned application is incorporated herein by reference.

### BACKGROUND

Current methods of detecting and treating cancer suffer from numerous limitations. Embodiments of the present disclosure address the aforementioned limitations.

### SUMMARY

In some embodiments, the present disclosure pertains to one or more peptoids. In some embodiments, the peptoids include, without limitation:

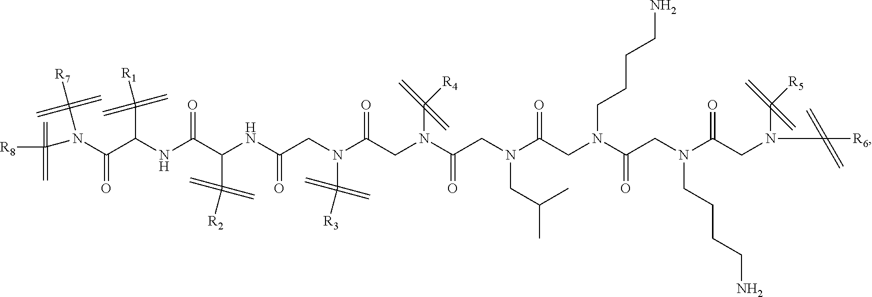

a multimer thereof, a derivative thereof, and combinations thereof. In some embodiments, R₁, R₂, R₃, R₄, R₅, R₆, R₇, and R₈ (R groups) each independently include, without limitation,

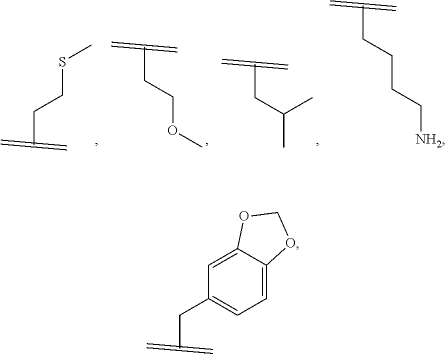

alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof.

In some embodiments, the peptoids of the present disclosure are suitable for use in treating or preventing a cancer in a subject. In some embodiments, the peptoids of the present disclosure are suitable for use in detecting a cancer in a subject. In some embodiments, the peptoids of the present disclosure are suitable for use as research and development tools.

Additional embodiments of the present disclosure pertain to methods of inhibiting the growth of cancer cells by exposing the cancer cells to the peptoids of the present disclosure. In various embodiments, the exposing occurs in vitro or in vivo.

Further embodiments of the present disclosure pertain to methods of treating or preventing a cancer in a subject by administering the peptoids of the present disclosure to the subject. Additional embodiments of the present disclosure pertain to methods of detecting cancer in a subject by exposing cells susceptible of being cancerous to the peptoids of the present disclosure, detecting the presence or absence of vimentin associated with cells susceptible of being cancerous, and correlating the presence or absence of the vimentin to the presence or absence of the cancer in the subject. In some embodiments, the presence of the vimentin is correlated to the presence of the cancer and the absence of the vimentin is correlated to the absence of the cancer.

### DESCRIPTION OF THE DRAWINGS

FIG. 1A illustrates a method of inhibiting the growth of cancer cells.

FIG. 1B illustrates a method of treating or preventing a cancer in a subject.

FIG. 1C illustrates a method of detecting a cancer in a subject.

FIG. 2 provides a model of malignant transformation of in vitro primary human bronchial epithelial cells (HBECs) following stepwise introduction of common lung cancer mutations. Step 1: HBEC were first immortalized by overexpressing cdK4 and hTERT to obtain HBEC3-KT cells. Step 2: The p53 gene was inactivated in HBEC3-KT cells through knockdown to obtain HBEC3^{p53} cells. Step 3: After p53 loss, KRAS overexpression was introduced to obtain HBEC3^{p53,KRAS} cells. Step 4: cMYC was overexpressed in HBEC3^{p53,KRAS} cells to result in epithelial-to-mesenchymal transition (EMT) and the attainment of HBEC3^{p53,KRAS,cMYC} cells.

FIGS. 3A and 3B provide comparative structures of peptides (FIG. 3A) and peptoids (FIG. 3B).

FIG. 4 illustrates a scheme for synthesizing peptoids.

FIGS. 5A-5C illustrate an assay for screening peptoids that bind to HBEC3^{p53,KRAS,cMYC} cells. FIG. 5A provides a pictorial representation of a one bead two color (OBTC) assay. The fully transformed HBEC3^{p53,KRAS,cMYC} cells (FIG. 2, step 4) were stained using red Q-dots and normal cells HBEC3-KT were stained in green using green Q-dots. One million cells of each color were mixed in 1:1 ratio and were incubated for 1 hour with beads (100,000 beads library) containing one-bead one-compound library at 23° C. FIG. 5B illustrates the identification of beads bound to red cells, which is binding to a receptor present only on transformed cells (HBEC3^{p53, KRAS,cMYC}) but absent in normal cell (HBEC3-KT). FIG. 5C provides a chemical structure of the screened peptoid JM3A, which binds to transformed cells.

FIGS. 6A-6C shows additional results related to the efficacy of JMSA in binding to HBEC3^{p53,KRAS,cMYC} cells. FIG. 6A shows HBEC3-KT cells staining in green with Qdot 565 and beads containing JM3A incubated with green cells. FIG. 6B shows HBEC3^{p53,KRAS,cMYC} cells stained in red with Qdot 655 and beads containing JM3A incubated with red cells. FIG. 6C shows a 1:1 mixture of green and red cells incubated with beads containing JM3A. Only red cells bound to beads in all situations, indicating the high specificity of JM3A to HBEC3^{p53,KRAS,cMYC} cells.

FIGS. 7A-7C shows results related to the binding of JM3A to HBEC3^{p53,KRAS,cMYC} cells. FIG. 7A shows the chemical structure of JM3A with biotin at the C-terminus and benzophenone at the N-terminus. FIG. 7B shows a magnetic bead JM3A pulldown of the target HBEC3^{p53,KRAS, cMYC} cells, where the bound proteins were separated by electrophoresis and visualized by silver staining.

FIG. 8 illustrates the presence of vimentin in both precancerous and normal cells around them.

FIGS. 9A-9D provide additional results related to the binding of JM3A to HBEC3^{p53,KRAS, cMYC} cells. FIG. 9A shows the beads containing JM3A after incubation with histidine-tagged vimentin followed by anti-His Alexa 647. The protein showed binding with on-bead JM3A. FIG. 9B shows a control experiment, where JM3A on tentagel beads were incubated with anti-His Alexa 647. The beads did not show any binding to the antibody. FIG. 9C shows another control experiment, where the vimentin protein was pre-incubated with 10×JM3A, which were then added to the beads. A reduced binding of vimentin with beads was observed, further affirming the specificity of the interaction of JM3A with vimentin. FIG. 9D shows the relative quantification of vimentin expression in various transformed cells, including HBEC3^{p53,KRAS,cMYC}

FIGS. 10A-10B show additional pull-down assays for JMSA. FIG. 10A shows a western blot analysis of magnetic-bead pulldown with JM3A and control PC462 on normal cells (HBEC3-KT) and transformed cells (HBEC3^{p53,KRAS,cMYC}). FIG. 10B shows a western blot analysis of magnetic-bead pulldown assay samples with commercially available vimentin protein using JM3A, PC462, and pre-incubated vimentin with JM3A/anti-vimentin antibody.

FIGS. 11A-D show additional characterizations of the binding of JMSA to vimentin in cells. FIG. 11A shows the results of a pulldown assay, where higher NAD (P) H signals suggest that vimentin-rich transformed cells (HBEC3^{p53,KRAS,cMYC}) were pulled down more in quantity than the normal cells (HBEC3-KT). FIG. 11B shows the results of a pulldown assay, where higher dityrosine signals suggest that vimentin-rich transformed cells (HBEC3^{p53,KRAS,cMYC}) were pulled down more in quantity than the normal cells (HBEC3-KT). FIG. 11C shows the results of a pulldown assay, where higher absorbance signal in the MTS assay suggests that vimentin-rich transformed cells (HBEC3^{p53,KRAS,cMYC}) were pulled down more in quantity than the normal cells (HBEC3-KT). FIG. 11D shows the immunohistochemical detection of vimentin overexpression by using JM3A (top panel) and vimentin antibody (bottom panel) in early cancer tissues (panels 1-4 and 6-9, present) and normal tissues (panels 5 and 10, absent).

FIGS. 12A-12D provide data related to the characterization of JMSA. FIG. 12A provides the structures of different JM3A derivatives, where different R groups were replaced with alanine/sarcosine. FIG. 12B provides an ELISA-like assay data showing the significance of the 5^{th}, 6^{th}, and 7^{th} residues in JM3A for binding to vimentin (JM3A-5, JM3A-6 and JM3A-7 derivatives lacked their original residues and had significantly reduced binding). FIG. 12C provides a structure of JM3A, where the essential and non-essential residues are differentially highlighted. FIG. 12D provides an ELISA-like binding quantification of JM3A binding to vimentin, showing a K_{d} of 19 μM.

FIGS. 13A-13B provide additional data related to the characterization of JMSA and its derivatives. FIG. 13A shows the chemical structure of JM3A-BP, where a benzophenone in attached to the N-terminus of the peptoid. FIG. 13B shows the results of an ELISA-like assay, demonstrating a significantly strong binding of JM3A-BP in comparison to all other derivatives.

FIGS. 14A-14E provide additional data related to the characterization of JMSA and its derivatives. FIG. 14A provides the chemical structure of JM3A-8-BP. FIG. 14B provides the chemical structure of JM3A-4,8-BP. FIG. 14C provides the chemical structure of JM3A-4-iso-8-BP. FIG. 14D provides an ELISA-like binding assay data of the JM3A derivatives. FIG. 14E provides a vimentin binding quantification for JM3A-4,8-BP, showing K_{d}=7 μM.

FIGS. 15A-15C provide data related to the characterization of JM3A-4,8-BP and its dimer (JM3A-4,8-BPD1). FIG. 15A provides the chemical structure of JM3A-4,8-BP. FIG. 15B provides the chemical structure of JM3A-4,8-BPD1 dimer. FIG. 15C provides an ELISA-like binding assay comparing dimer JM3AD1 to JM3A-4,8-BPD1 dimer.

FIGS. 16A-16D provide data related to the characterization of JM3A-4,8-BPD1. FIG. 16A provides the chemical structure of JM3A-4,8-BPD1. FIG. 16B provides the chemical structure of JM3A-4.8-BPD1-4. FIG. 16C provides an ELISA-like binding assay comparing binding affinity of dimer compounds. FIG. 16D provides the chemical structures of derivatives that showed significant binding affinity upon replacing the benzophenone moiety.

FIGS. 17A-17D provide data related to the characterization of JM3A-BPD1. FIG. 17A shows the structure of JM3A-BPD1 homo-dimer linker derivatives. L1, L2, L3, and L4 have one, two, three, and four glycines respectively added to the central lysine. L5 has a lysine as a central linker with two PEG linkers and two glycines. L6 and L7 have 4 PEG and 8PEG linkers with a lysine. FIG. 17B shows the structure of a control compound. FIG. 17C shows a standard MTS assay that was used to assess anti-proliferation activity of homo-dimer peptoids on H1299 cells. FIG. 17D shows the structure of L2.

FIGS. 18A-18D provide data related to the characterization of L2. FIG. 18A shows the structure of L2. FIG. 18B shows the structure of L2-(Ala). FIG. 18C shows a standard MTS assay that was used to assess anti-proliferation activity of homodimer peptoids on H1299 cells. FIG. 18D shows the structure of L2-(D-Ala).

FIGS. 19A-19B provide additional data related to the characterization of L2. FIG. 19A shows concentration-dependent binding curves of JM3A-BPD1 and L2 (D-ala) evaluated using ELISA-like binding assay. FIG. 19B shows the K_{d} value of JM3A-BPD1 and L2 (D-ala) for binding to vimentin.

FIGS. 20A-20B provide additional data related to the characterization of L2. FIG. 20A shows a standard MTS assay of L2 (D-ala) on various lung cancer models. FIG. 20B shows an IC₅₀ value of L2 (D-ala) treatment on different lung cancer models with various vimentin expression levels.

FIGS. 21A-21D provide a time-course MTS assays of L2 on H1299 cells.

FIGS. 22A-22B provide data related to the characterization of L2 (D-ala), JM3A-BPD1, and JM3A-BP. FIG. 22A shows the inhibition of cell migration by L2 (D-ala), JM3A-BPD1, JM3A-BP and control compounds. H1299 cells were cultured to form a monolayer in 24-well plate. A scratch was made and wells were treated with L2 (D-ala), JM3A-BPD1. JM3A-BP, and control compounds in a concentration between 300 nM to 1 μM. FIG. 22B shows the quantification of the distance of the scratch area.

FIGS. 23A-23B provide additional data related to the characterization of L2 (D-ala), JM3A-BPD1, and JM3A-BP. FIG. 23A shows H460 cells that were sparsely seeded and treated with L2 (D-ala), JM3A-BPD1, JM3A-BP, and control compound on day 1 and incubated for 7 days. FIG. 23B provides a quantification of the colonies after a 7-day incubation treatment.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Early diagnosis of cancer is the key to achieve better patient survival in cancer. For instance, the delayed diagnosis of cancer at mid-to-late stages limits the efficacy of conventional anticancer treatment approaches, such as surgical resection, chemotherapy, radiotherapy, and targeted therapies.

Moreover, despite the past decade's progress in cancer treatments, patient survival has improved only marginally in many cancer types. For instance, non-small cell lung cancer (NSCLC) remains the leading cause of cancer deaths in the US and world-wide. In particular, the 5-year survival rates for NSCLC range from 60% for stage IA to 5% for stage IV. This high mortality is primarily due to late diagnosis of locally advanced or metastatic disease as tumor heterogeneity limits treatment options at that point.

Additionally, invasive biopsies are needed to confirm most cancers. However, such biopsies are painful, costly and limited by adequate sampling.

Furthermore, most of the nascent cancers are symptomless and current imaging modalities are less accurate with high rates of false positives or negatives. For instance, the current state-of-the-art diagnostic technologies, such as ultrasound, Positron Emission Tomography-Computed Tomography (PET-CT) and Magnetic Resonance Imaging (MRI), have numerous instrumental limitations.

Targeted molecular imaging and therapy are considered as the best approaches for early detection and better management of cancer. However, the paucity of reliable biomarkers that express in the earliest stages of cancer present a major challenge for developing high sensitive imaging probes and effective therapies.

Accordingly, a need exists for high affinity ligands to non-invasively detect cancer biomarkers. A need also exists for stable and high affinity ligands to treat cancers, such as early stage malignant cancers. Numerous embodiments of the present disclosure address the aforementioned needs.

### Peptoids

In some embodiments, the present disclosure pertains to peptoids. In some embodiments, the peptoids of the present disclosure are suitable for use in treating or preventing a cancer in a subject. In some embodiments, the peptoids of the present disclosure are suitable for use in detecting a cancer in a subject. In some embodiments, the peptoids of the present disclosure are suitable for use as research and development tools.

In some embodiments, the peptoids of the present disclosure include, without limitation one or more of the following structures:

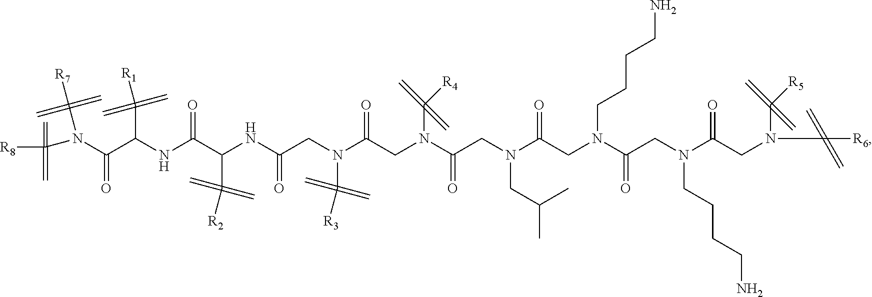

a multimer thereof, a derivative thereof, and combinations thereof.

In some embodiments, R₁, R₂, R₃, R₄, R₅, R₆, R₇, and R₈ (R groups) each independently include, without limitation, the following groups:

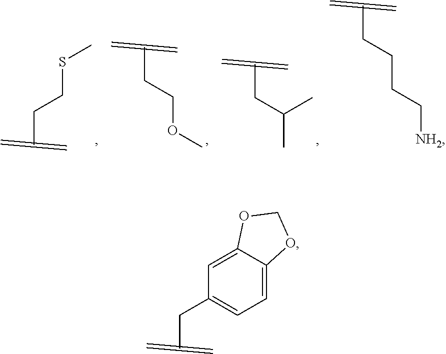

alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof.

In some embodiments, the peptoids of the present disclosure include, without limitation, one or more of the following structures:

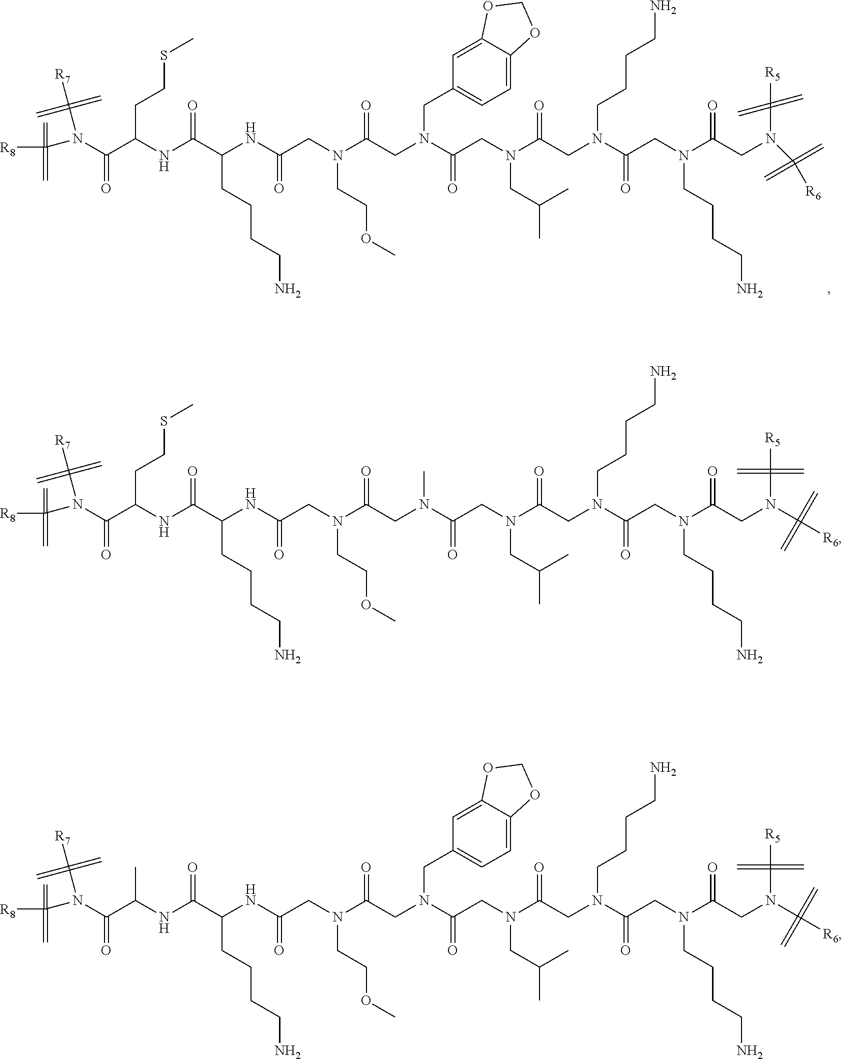

a multimer thereof, a derivative thereof, and combinations thereof.

In some embodiments, the peptoids of the present disclosure include, without limitation, one or more of the following structures:

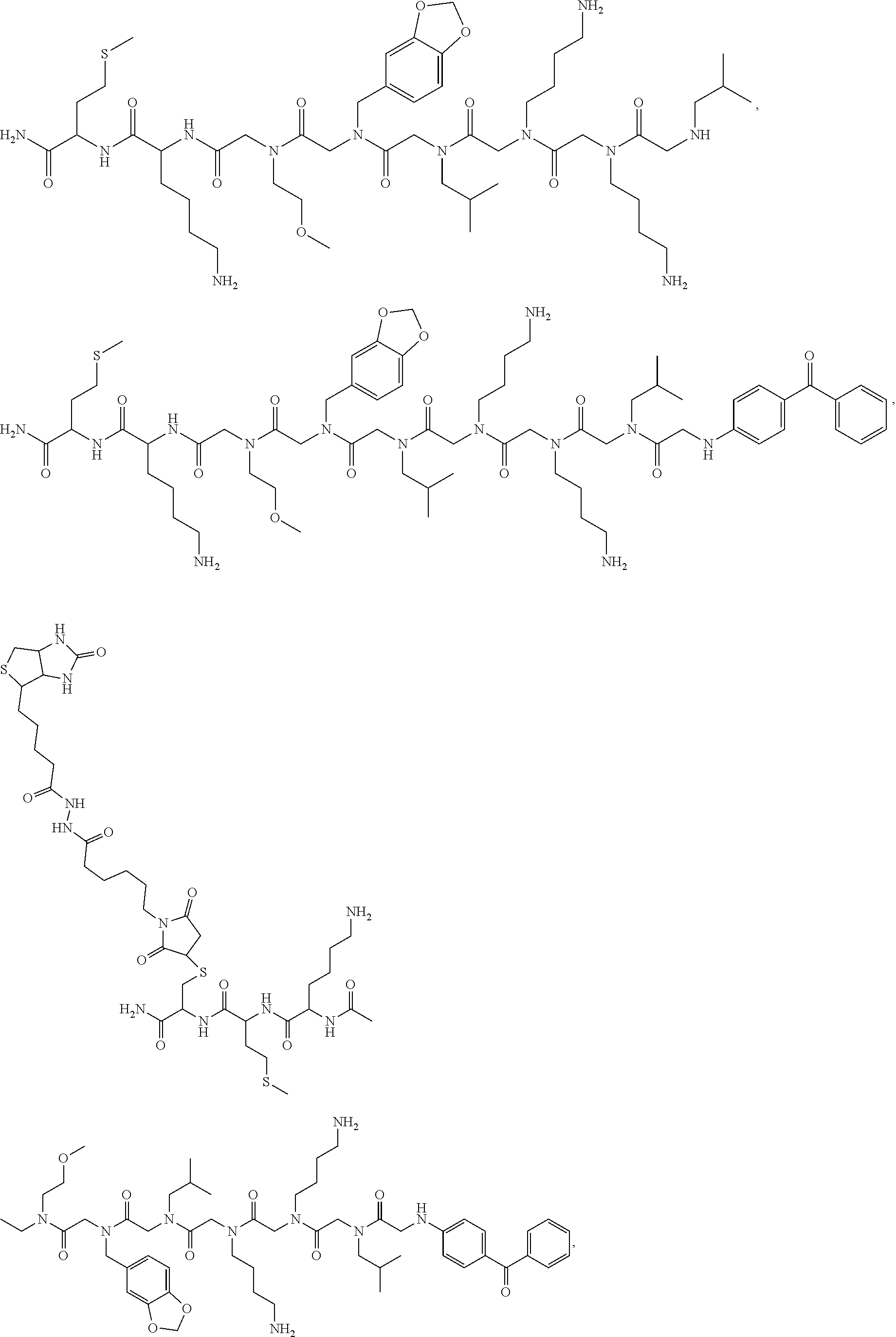

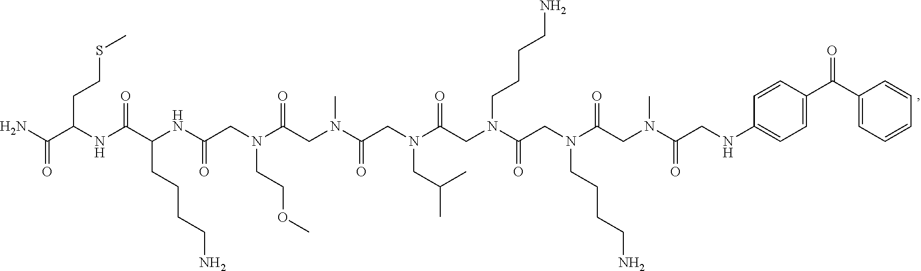 [SMILES: COCCN(CC(=O)NC(CCCCN)C(=O)NC(CCSC)C(N)=O)C(=O)CN(C)C(=O)CN(CC(C)C)C(=O)CN(CCCCN)C(=O)CN(CCCCN)C(=O)CN(C)C(=O)CNc1ccc(C(=O)c2ccccc2)cc1]

multimers thereof, derivatives thereof, and combinations thereof.

In some embodiments, the peptoids of the present disclosure include the following structure:

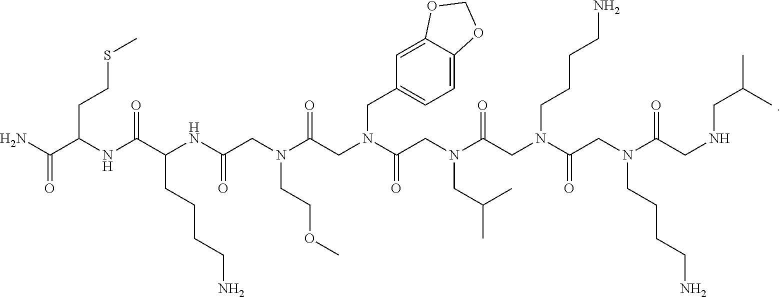 [SMILES: COCCN(CC(=O)NC(CCCCN)C(=O)NC(CCSC)C(N)=O)C(=O)CN(Cc1ccc2c(c1)OCO2)C(=O)CN(CC(C)C)C(=O)CN(CCCCN)C(=O)CN(CCCCN)C(=O)CNCC(C)C]

In some embodiments, the peptoids of the present disclosure include the following structure:

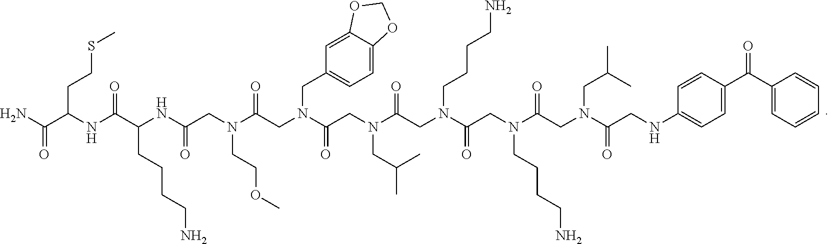 [SMILES: COCCN(CC(=O)NC(CCCCN)C(=O)NC(CCSC)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1]

In some embodiments, the peptoids of the present disclosure include the following structure:

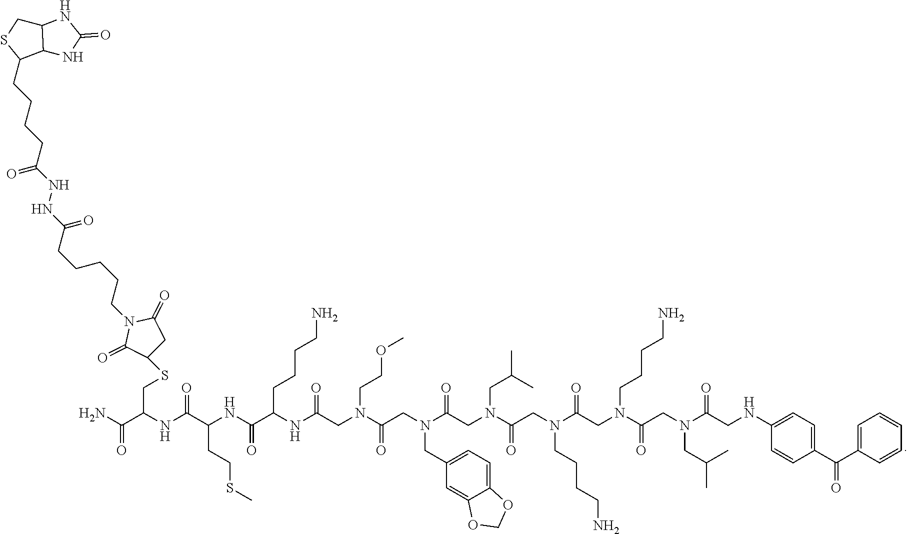 [SMILES: COCCN(CC(=O)NC(CCCCN)C(=O)NC(CCSC)C(=O)NC(CSC1CC(=O)N(CCCCCC(=O)NNC(=O)CCCCC2SCC3NC(=O)NC32)C1=O)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1]

In some embodiments, the peptoids of the present disclosure include the following structure:

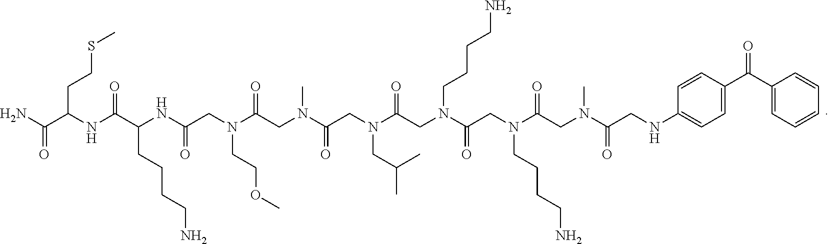 [SMILES: COCCN(CC(=O)NC(CCCCN)C(=O)NC(CCSC)C(N)=O)C(=O)CN(C)C(=O)CN(CC(C)C)C(=O)CN(CCCCN)C(=O)CN(CCCCN)C(=O)CN(C)C(=O)CNc1ccc(C(=O)c2ccccc2)cc1]

### Multimers

The peptoids of the present disclosure may be in various forms. For instance, in some embodiments, the peptoids of the present disclosure are in the form of a monomer. In some embodiments, the peptoids of the present disclosure are in the form of a multimer. In some embodiments, the multimer includes, without limitation, a homomultimer, a heteromultimer, a cyclic multimer, a dimer, a trimer, a tetramer, and combinations thereof.

The peptoids of the present disclosure may be connected to one another in various forms to form multimers. For instance, in some embodiments, the peptoids in the multimer are connected through one or more covalent linkages on peptoid backbones, R groups, and combinations thereof.

Additionally, such covalent linkages may be positioned at various positions of peptoids. For instance, in some embodiments, the covalent linkages are positioned at the C-terminus of peptoids, the N-terminus of peptoids, regions proximal to the N-terminus of peptoids, middle regions of peptoids, regions proximal to the C-terminus of peptoids, and combinations thereof.

In some embodiments, the covalent linkages include one or more of the R groups. In some embodiments, the covalent linkages include the R₇ group, the R₈ group, and combinations thereof.

In some embodiments, the covalent linkages include one or more linkers. In some embodiments, the one or more linkers include, without limitation, rigid linkers, semi-rigid linkers, flexible linkers, semi-flexible linkers, cleavable linkers, non-cleavable linkers, lysine-based linkers, glycine-based linkers, cyclic linkers, heterocyclic linkers, alicyclic linkers, non-cyclic linkers, aliphatic linkers, aromatic linkers, sulfide-based linkers, ester-based linkers, ether-based linkers, polyethylene glycol-based linkers, glycol-based linkers, allyl-based linkers, benzyl-based linkers, amino hexanoic-based linkers, NHS ester-based linkers, maleimide-based linkers, and combination thereof.

In some embodiments, the multimeric peptoids of the present disclosure include, without limitation, one or more of the following structures:

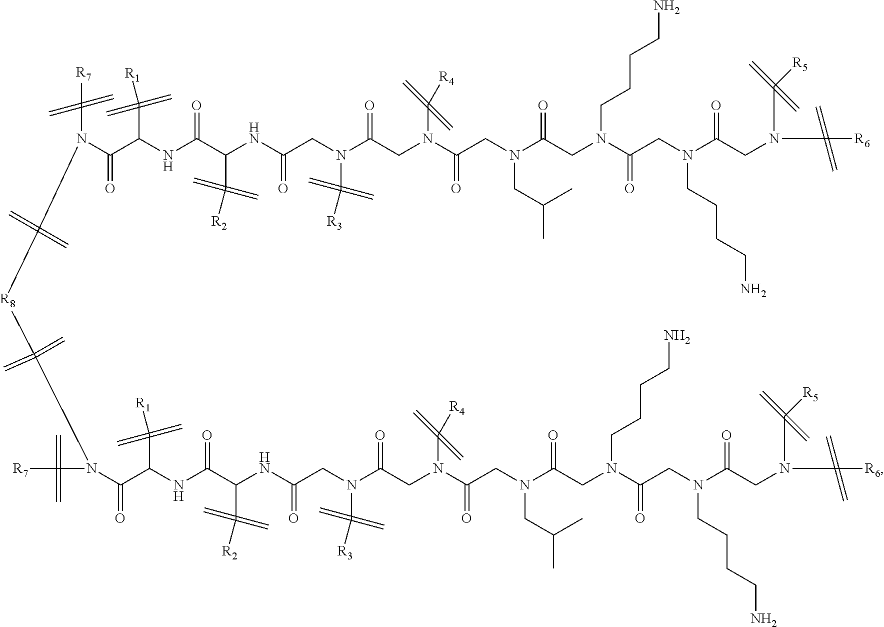

a derivative thereof, and combinations thereof.

In some embodiments, the multimeric peptoids of the present disclosure include, without limitation, one or more of the following structures:

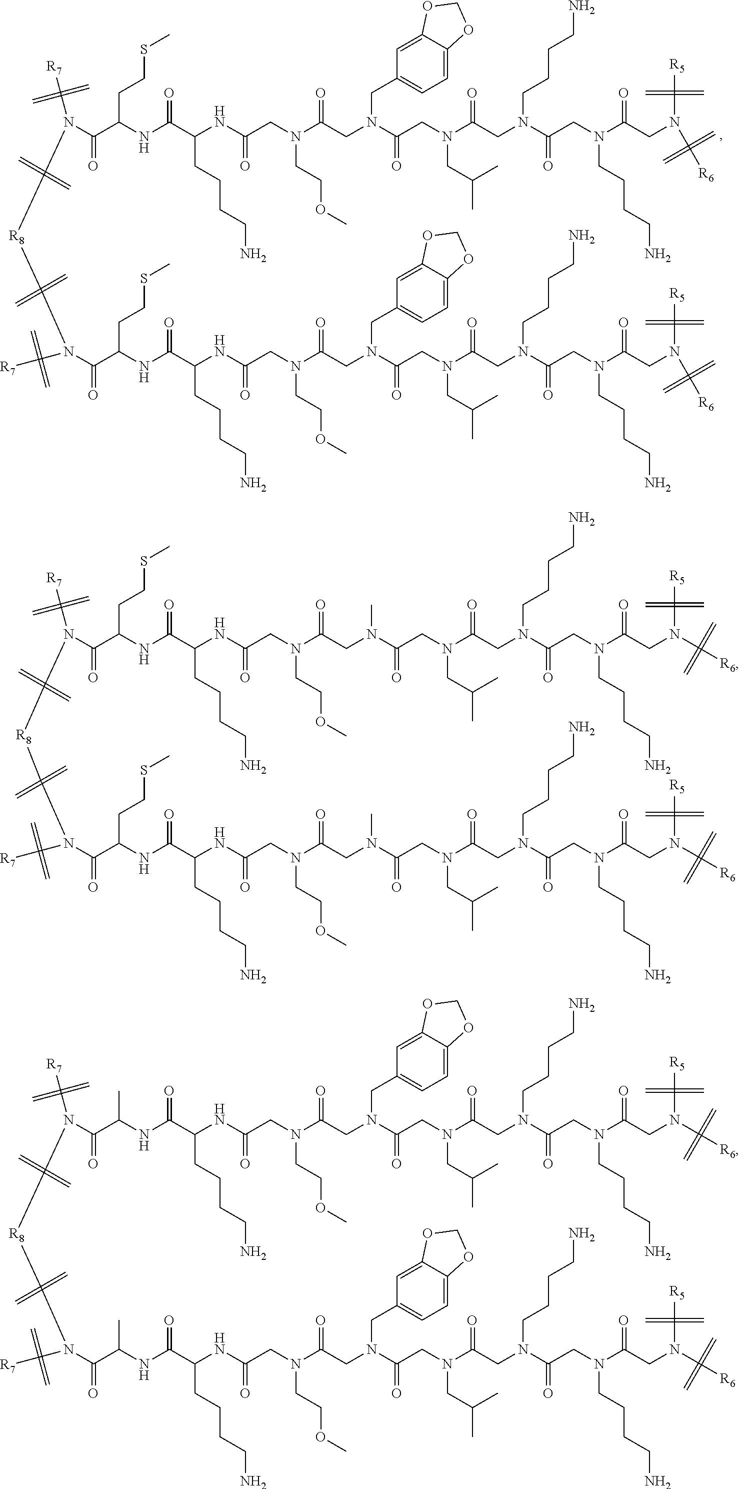

derivatives thereof, and combinations thereof.

In some embodiments, the peptoids of the present disclosure include one or more of the following structures:

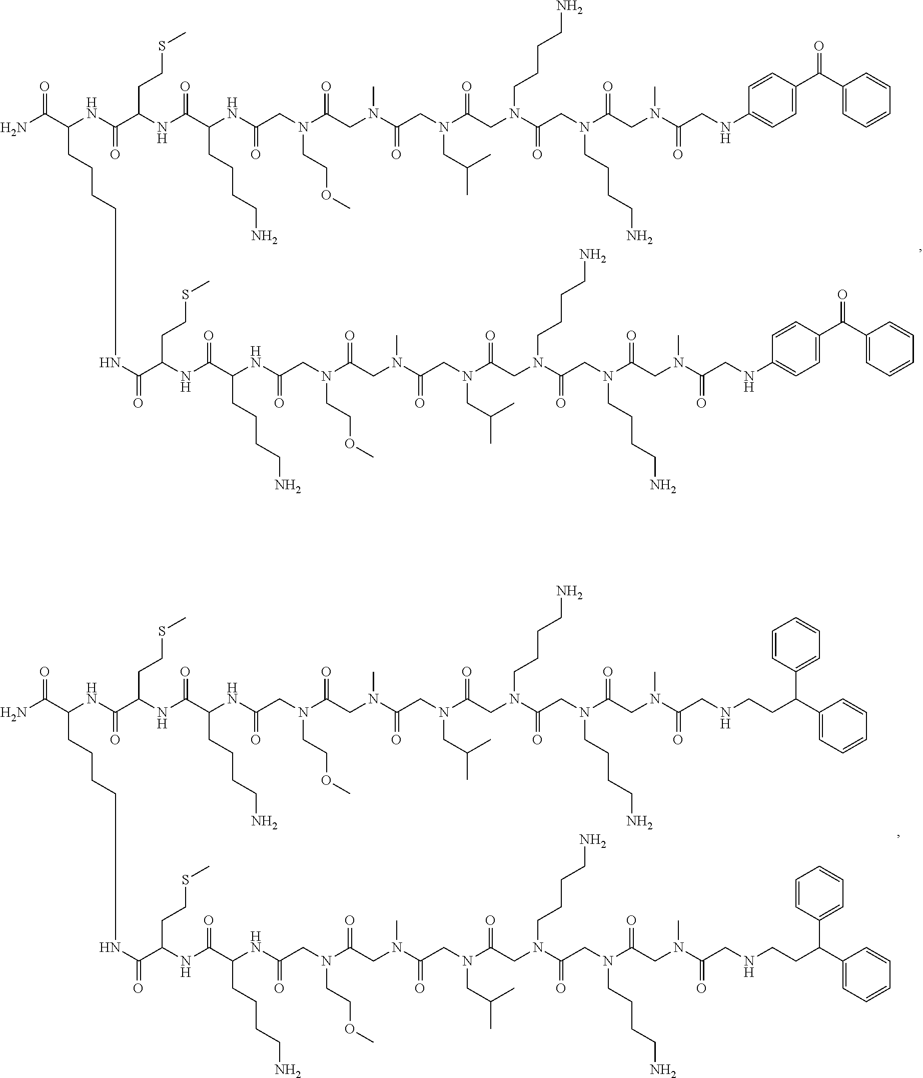

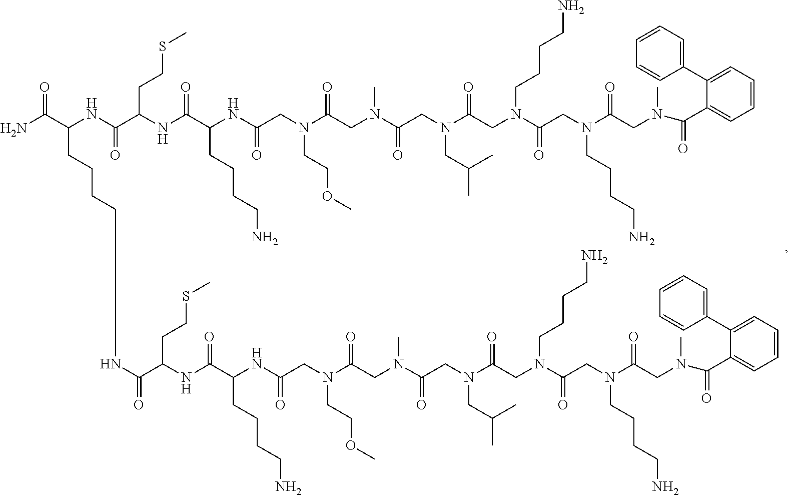 [SMILES: COCCN(CC(=O)NC(CCCCN)C(=O)NC(CCSC)C(=O)NCCCCC(NC(=O)C(CCSC)NC(=O)C(CCCCN)NC(=O)CN(CCOC)C(=O)CN(C)C(=O)CN(CC(C)C)C(=O)CN(CCCCN)C(=O)CN(CCCCN)C(=O)CN(C)C(=O)c1ccccc1-c1ccccc1)C(N)=O)C(=O)CN(C)C(=O)CN(CC(C)C)C(=O)CN(CCCCN)C(=O)CN(CCCCN)C(=O)CN(C)C(=O)c1ccccc1-c1ccccc1]

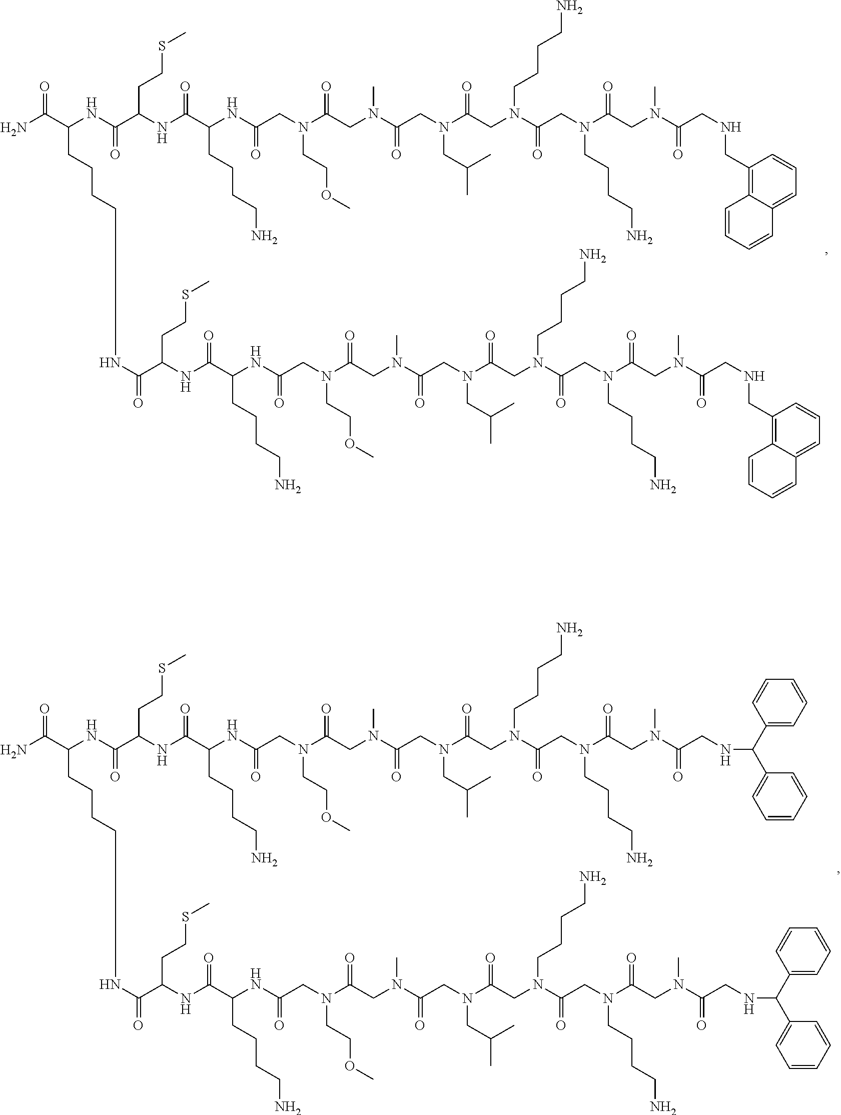

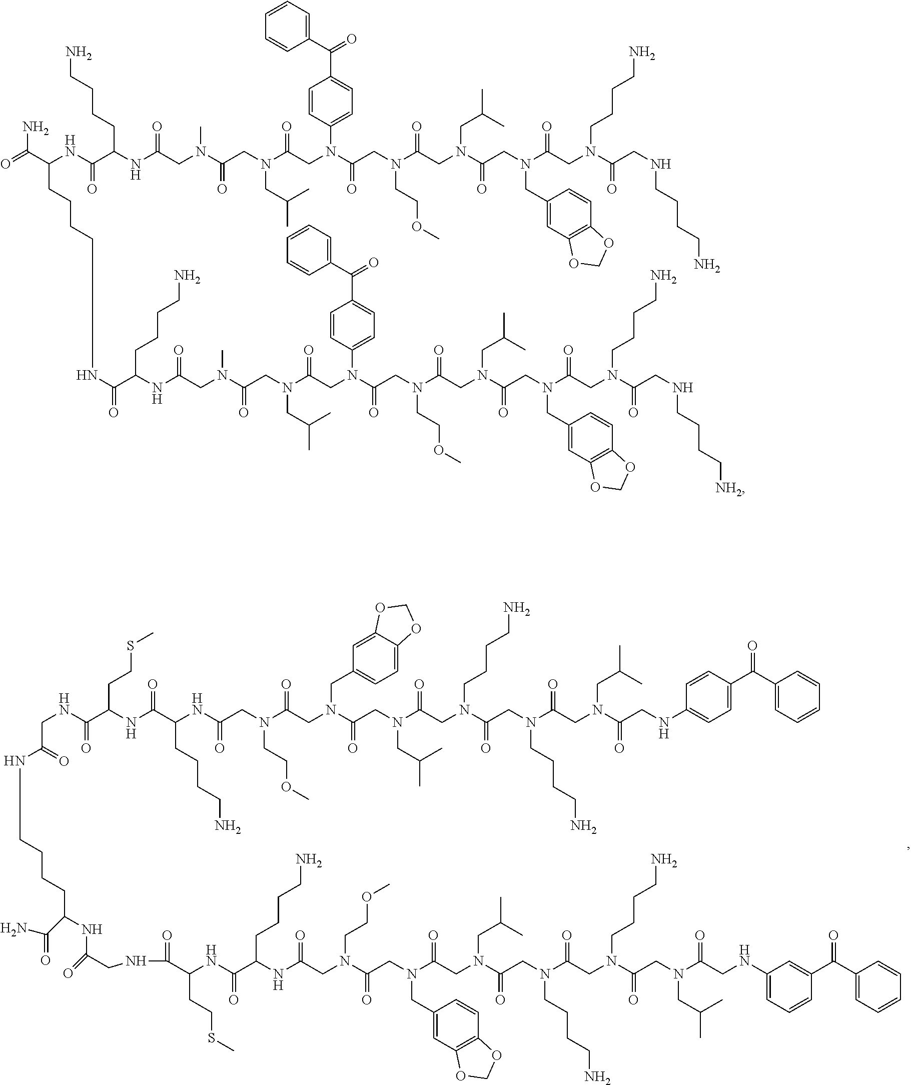

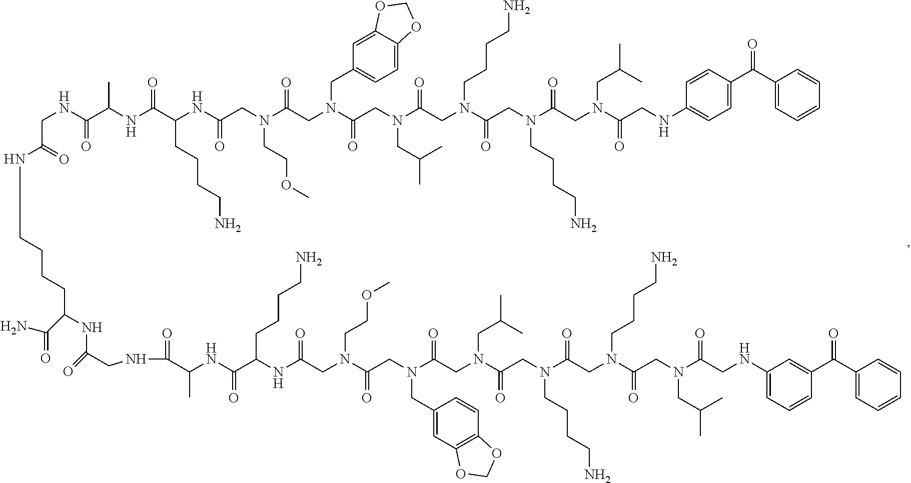 [SMILES: COCCN(CC(=O)NC(CCCCN)C(=O)NC(C)C(=O)NCC(=O)NCCCCC(NC(=O)CNC(=O)C(C)NC(=O)C(CCCCN)NC(=O)CN(CCOC)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1cccc(C(=O)c2ccccc2)c1)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1]

derivatives thereof, and combinations thereof.

In some embodiments, the peptoids of the present disclosure include the following structure:

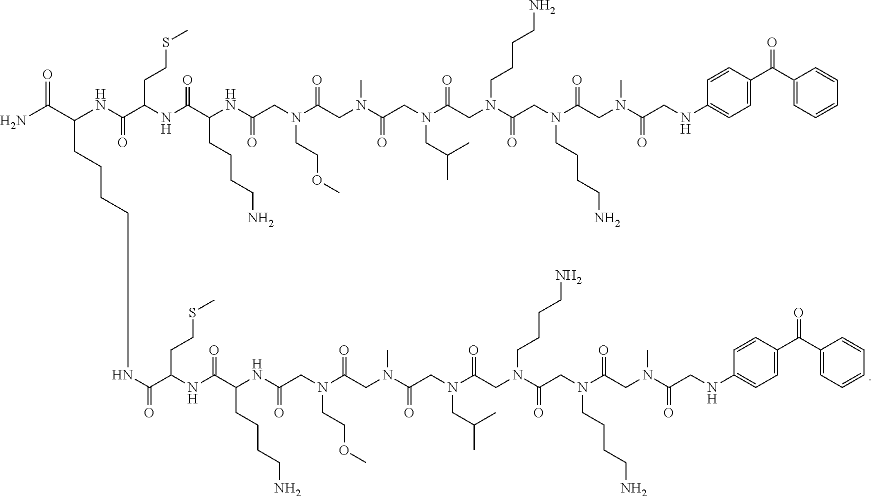 [SMILES: COCCN(CC(=O)NC(CCCCN)C(=O)NC(CCSC)C(=O)NCCCCC(NC(=O)C(CCSC)NC(=O)C(CCCCN)NC(=O)CN(CCOC)C(=O)CN(C)C(=O)CN(CC(C)C)C(=O)CN(CCCCN)C(=O)CN(CCCCN)C(=O)CN(C)C(=O)CNc1ccc(C(=O)c2ccccc2)cc1)C(N)=O)C(=O)CN(C)C(=O)CN(CC(C)C)C(=O)CN(CCCCN)C(=O)CN(CCCCN)C(=O)CN(C)C(=O)CNc1ccc(C(=O)c2ccccc2)cc1]

In some embodiments, the peptoids of the present disclosure include the following structure:

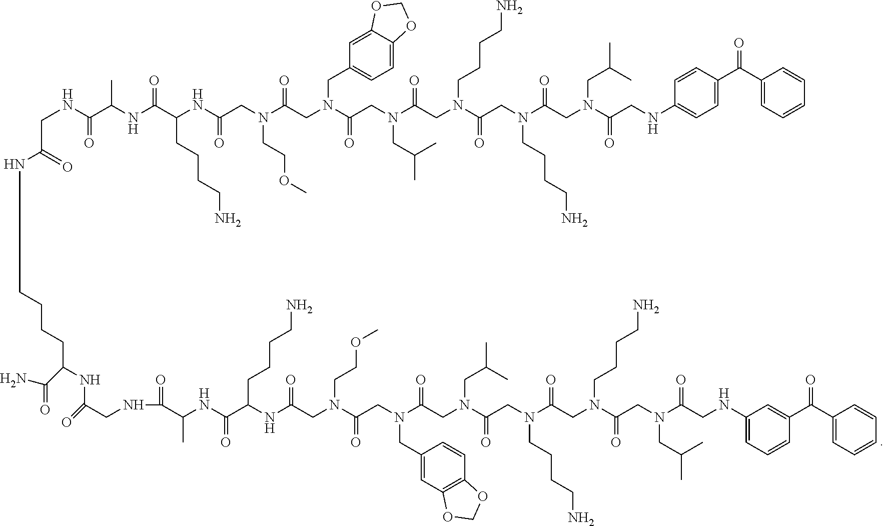 [SMILES: COCCN(CC(=O)NC(CCCCN)C(=O)NC(C)C(=O)NCC(=O)NCCCCC(NC(=O)CNC(=O)C(C)NC(=O)C(CCCCN)NC(=O)CN(CCOC)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1cccc(C(=O)c2ccccc2)c1)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1]

In some embodiments, the peptoids of the present disclosure include the following structure:

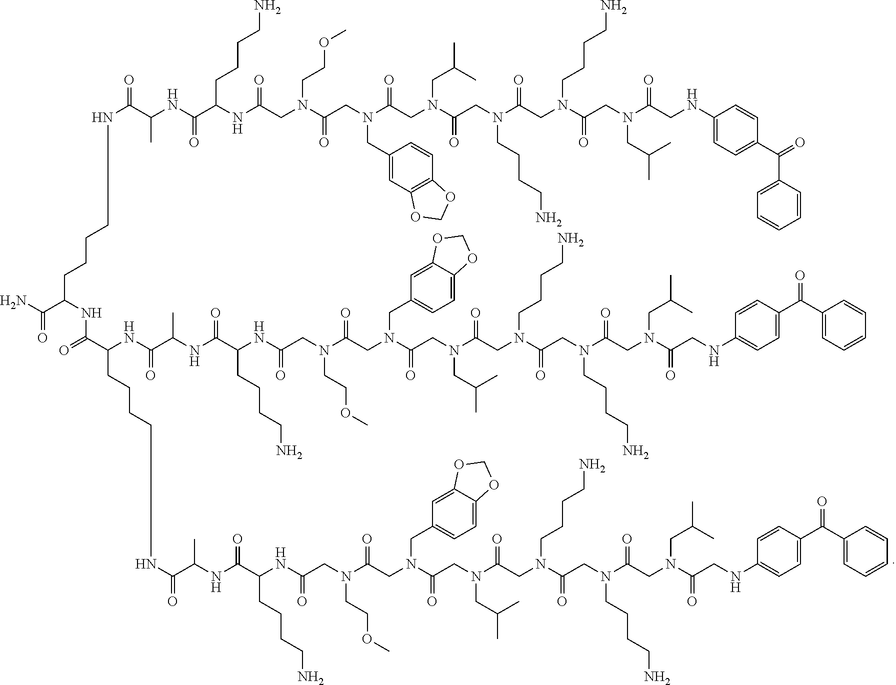 [SMILES: COCCN(CC(=O)NC(CCCCN)C(=O)NC(C)C(=O)NCCCCC(NC(=O)C(CCCCNC(=O)C(C)NC(=O)C(CCCCN)NC(=O)CN(CCOC)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1)NC(=O)C(C)NC(=O)C(CCCCN)NC(=O)CN(CCOC)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1]

In some embodiments, the peptoids of the present disclosure include the following structure:

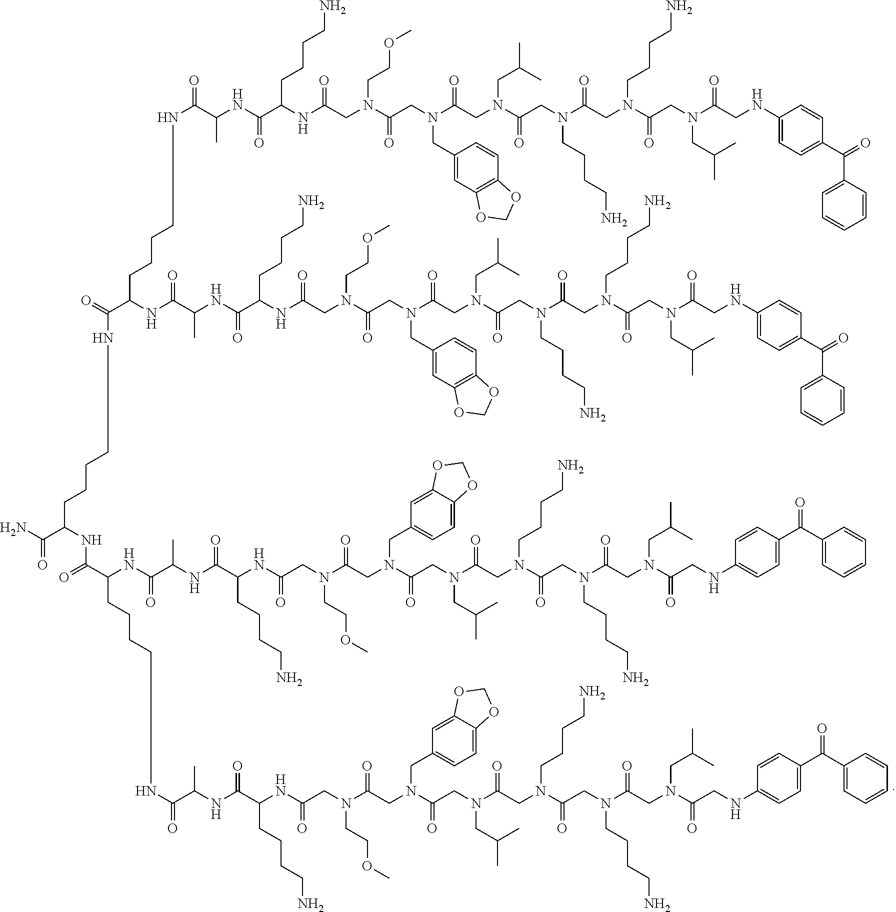

In some embodiments, the peptoids of the present disclosure include one or more peptoid derivatives. In some embodiments, the one or more peptoid derivatives include one or more peptoid moieties derivatized with one or more functional groups. In some embodiments, the one or more peptoid moieties are positioned on peptoid backbones, R groups, and combinations thereof. In some embodiments, the functional groups include, without limitation, alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof.

### Tracing Agents

In some embodiments, the peptoids of the present disclosure are associated with one or more tracing agents. In some embodiments, the tracing agents are utilized to detect the association of the peptoids of the present disclosure with cells, such as cancer cells.

Tracing agents may be associated with the peptoids of the present disclosure in various manners. For instance, in some embodiments, the tracing agents of the present disclosure are linked to or represented by at least one of the R groups of the peptoids of the present disclosure. In some embodiments, the tracing agents of the present disclosure are linked to or represented by the R₅ group, the R₆ group, and combinations thereof.

The peptoids of the present disclosure may include various types of tracing agents. For instance, in some embodiments, the tracing agent includes, without limitation, fluorophores, chromophores, dyes, radio-labeled molecules, radioactive nuclei, high contrast agents, gadolinium, gallium, thallium, fluorinated compounds, biotin, biotinylated compounds, phenyl-based tracing agents, biphenyl-based tracing agents, benzophenone-based tracing agents, polycyclic aromatic tracing agents, photoaffinity labeling agents (e.g. benzophenones), drugs (e.g. chemotherapeutics), and combinations thereof.

### Peptoid Forms

The peptoids of the present disclosure may be in various forms. For instance, in some embodiments, the peptoids of the present disclosure are within a composition. In some embodiments, the composition is in a form that includes, without limitation, nasal sprays, eye drops, injectable suspensions, tablets, and combinations thereof.

### Methods of Inhibiting the Growth of Cancer Cells

The peptoids of the present disclosure may be suitable for use in inhibiting the growth of cancer cells. As such, in additional embodiments, the present disclosure pertains to methods of inhibiting the growth of cancer cells.

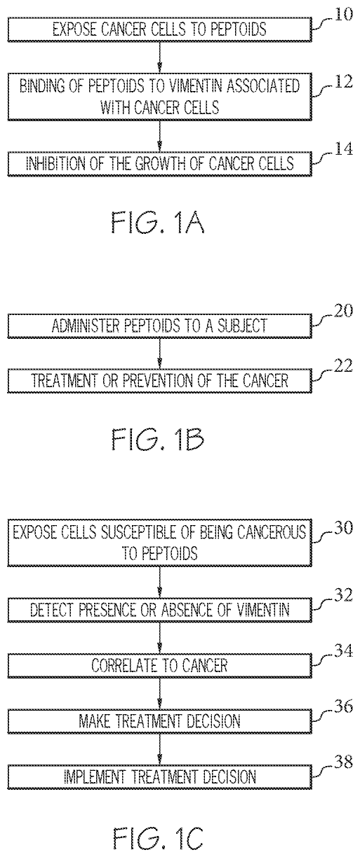

In some embodiments illustrated in FIG. 1A, the methods of the present disclosure include exposing the cancer cells to the peptoids of the present disclosure (step 10). In some embodiments, the exposing results in the binding of the peptoids to vimentin associated with the cancer cells (step 12). This in turn results in the inhibition of the growth of the cancer cells (step 14). As set forth in more detail herein, the methods of the present disclosure can have numerous embodiments.

### Cancer Cells

The methods of the present disclosure may be utilized to inhibit the growth of various types of cancer cells. For instance, in some embodiments, the cancer cells include cancer cells that express vimentin. In some embodiments, the cancer cells include, without limitation, lung cancer cells, non-small cell lung cancer cells, colon cancer cells, esophageal cancer cells, breast cancer cells, prostate cancer cells, melanoma cells, cervical cancer cells, and combinations thereof. In some embodiments, the cancer cells include lung cancer cells. In some embodiments, the cancer cells include non-small cell lung cancer cells.

### Vimentin

The peptoids of the present disclosure may bind to vimentin associated with cancer cells. Vimentin may be positioned at various regions of cancer cells. For instance, in some embodiments, vimentin is positioned on the surface of the cancer cells.

In some embodiments, vimentin includes SEQ ID NO: 1 or a sequence with at least 60% sequence identity to SEQ ID NO: 1. In some embodiments, vimentin includes a sequence with at least 65% sequence identity to SEQ ID NO: 1. In some embodiments, vimentin includes a sequence with at least 70% sequence identity to SEQ ID NO: 1. In some embodiments, vimentin includes a sequence with at least 75% sequence identity to SEQ ID NO: 1. In some embodiments, vimentin includes a sequence with at least 80% sequence identity to SEQ ID NO: 1. In some embodiments, vimentin includes a sequence with at least 85% sequence identity to SEQ ID NO: 1. In some embodiments, vimentin includes a sequence with at least 90% sequence identity to SEQ ID NO: 1. In some embodiments, vimentin includes a sequence with at least 95% sequence identity to SEQ ID NO: 1. In some embodiments, vimentin includes a sequence with at least 99% sequence identity to SEQ ID NO: 1.

### Inhibition of the Growth of Cancer Cells

The peptoids of the present disclosure may inhibit the growth of cancer cells through various mechanisms. For instance, in some embodiments, the inhibition occurs through cancer cell death. In some embodiments, the inhibition occurs through reducing or eliminating proliferation of the cancer cells.

### Exposing of Peptoids to Cancer Cells

The peptoids of the present disclosure may be exposed to cancer cells in various manners. For instance, in some embodiments, the exposing occurs in vitro. In some embodiments, the exposing occurs in vivo in a subject.

In some embodiments, the exposing includes administering the peptoids of the present disclosure to a subject. In some embodiments, the administering occurs by a method that includes, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, and combinations thereof.

### Methods of Treating or Preventing Cancer in a Subject

In some embodiments, the peptoids of the present disclosure may be suitable for use in treating or preventing a cancer in a subject. As such, additional embodiments of the present disclosure pertain to methods of treating or preventing a cancer in a subject. In some embodiments illustrated in FIG. 1B, the methods of the present disclosure include administering to the subject a peptoid of the present disclosure (step 20) to result in the treatment or prevention of the cancer in the subject (step 22). As set forth in more detail herein, the treatment and prevention methods of the present disclosure can also have numerous embodiments.

### Administration of Peptoids

Various methods may be utilized to administer the peptoids of the present disclosure to subjects. For instance, in some embodiments, the administration occurs by methods that include, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, and combinations thereof.

### Treatment or Prevention of Cancers

In some embodiments, the methods of the present disclosure may be utilized to treat various types of cancers. In some embodiments, the methods of the present disclosure may be utilized to prevent various types of cancers. In some embodiments, the methods of the present disclosure may be utilized to treat and prevent various types of cancers.

The methods of the present disclosure may be utilized to treat or prevent various types of cancers. For instance, in some embodiments, the cancer includes, without limitation, lung cancer, non-small cell lung cancer, colon cancer, esophageal cancer, breast cancer, melanoma, prostate cancer, cervical cancer, and combinations thereof. In some embodiments, the cancer is lung cancer. In some embodiments, the cancer is non-small cell lung cancer.

### Subjects

The methods of the present disclosure may be utilized to treat or prevent cancers in various subjects. For instance, in some embodiments, the subjects include human beings. In some embodiments, the subjects may be suffering from a cancer. In some embodiments, the subjects may be vulnerable to a cancer. In some embodiments, the subjects include human beings suffering from a cancer.

### Methods of Detecting Cancers

The peptoids of the present disclosure may be suitable for use in detecting various types of cancers. Additional embodiments of the present disclosure pertain to methods of detecting cancer in a subject. In some embodiments illustrated in FIG. 1C, the methods of the present disclosure include exposing cells susceptible of being cancerous to the peptoids of the present disclosure (step 30), detecting the presence or absence of vimentin on cells susceptible of being cancerous (step 32), and correlating the presence or absence of the vimentin to the presence or absence of the cancer in the subject (step 34). In some embodiments, the presence of the vimentin is correlated to the presence of the cancer and the absence of the vimentin is correlated to the absence of the cancer. In some embodiments, the methods of the present disclosure also include a step of making a treatment decision (step 36). In some embodiments, the methods of the present disclosure also include a step of implementing the treatment decision (step 38). As set forth in more detail herein, the cancer detection methods of the present disclosure can have numerous embodiments.

### Exposing

Various methods may be utilized to expose cells susceptible of being cancerous to the peptoids of the present disclosure. For instance, in some embodiments, the exposing occurs in vitro. In some embodiments, the exposing further includes a step of isolating the cells from the subject and exposing the cells to the peptoids of the present disclosure in vitro.

In some embodiments, the exposing occurs in vivo. In some embodiments, the exposing includes administering the peptoid to the subject in vivo. In some embodiments, the administering occurs by a method that includes, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, and combinations thereof.

### Detecting

Various methods may be utilized to detect the presence or absence of vimentin on cells susceptible of being cancerous. For instance, in some embodiments, the detecting occurs by a method that includes, without limitation, visualization, microscopy, dark field microscopy, spectrometry, spectroscopy, colorimetric analysis, localized surface plasmon resonance (LSPR), nuclear magnetic resonance (NMR), computed tomography (CT), positron emission tomography (PET), surface plasmon resonance, electrochemistry, immunodetection, and combinations thereof.

In some embodiments, the detecting includes visualizing a color or image change of the cells. In some embodiments, the detecting occurs in a quantitative, semi quantitative, or qualitative manner.

### Cancers

The detection methods of the present disclosure may be utilized to detect various types of cancers. For instance, in some embodiments, the cancer includes, without limitation, lung cancer, non-small cell lung cancer, colon cancer, esophageal cancer, breast cancer, melanoma, prostate cancer, cervical cancer, and combinations thereof. In some embodiments, the cancer includes lung cancer. In some embodiments, the cancer includes non-small cell lung cancer.

### Subjects

The detection methods of the present disclosure may be utilized to detect cancer in various subjects. Suitable subjects were described previously. For instance, in some embodiments, the subject is a human being suffering from a cancer.

### Treatment Decisions

In some embodiments, the methods of the present disclosure also include a step of making a treatment decision. For instance, in some embodiments where the presence of vimentin is detected, the treatment decision includes treatment of the cancer. In some embodiments where vimentin is not detected, the treatment decision includes monitoring the subject. In some embodiments, the methods of the present disclosure also include a step of implementing the treatment decision.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Vimentin Targeted Peptoids for Early Diagnosis and Treatment of Cancer

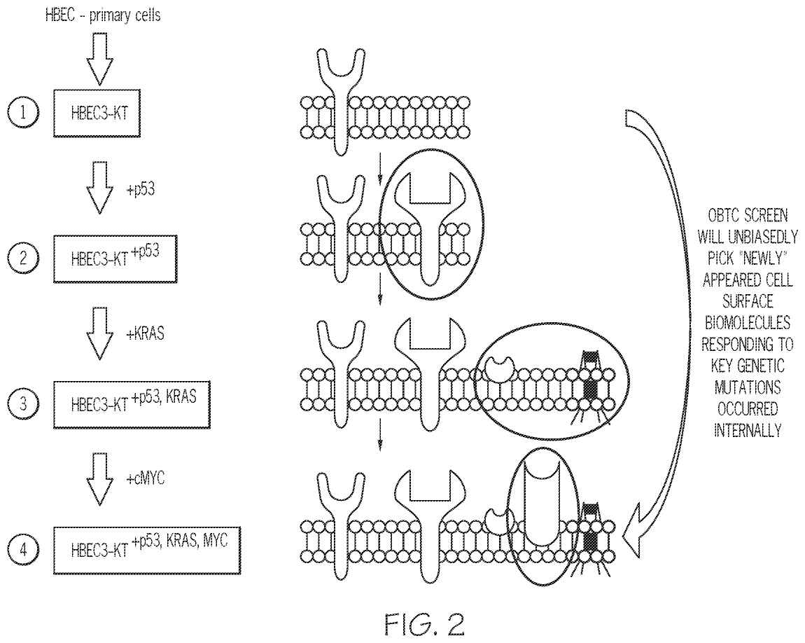

Human lung cancer development is a multistep process of specific proto-oncogene and tumor suppressor gene alterations in the cells. Multiple genetic and epigenetic alterations have been identified in genome-wide analysis of in lung tumors. It is crucial to identify which oncogenic alterations are responsible for tumor initiation and progression, which can become reliable targets for early detection and therapy. In recent years, a protocol has been established to transform primary human bronchial epithelial cells (HBECs) to full malignancy. Sato, M. et al., 2013. Human lung epithelial cells progressed to malignancy through specific oncogenic manipulations. Molecular cancer research: MCR, 11 (6). 638-650. This protocol involves sequential introduction of key oncogenic alterations: p53, KRAS, and cMYC in these cells (FIG. 2) that progress them to malignancy in a stepwise manner, resulting in their immortalization and transformation into fully malignant cells. The cell lines created at each step (named HBEC3-KT, HBEC3^{p53}, HBEC3^{p53,KRAS}, and HBEC3^{p53,KRAS,cMYC}) are expected to express genuine early biomarkers on their cell surface. We captured and identified them with a high specific peptoid to target those biomarkers.

### Example 1.1. Peptoids as Better Imaging Probe and Drug Delivering Agents

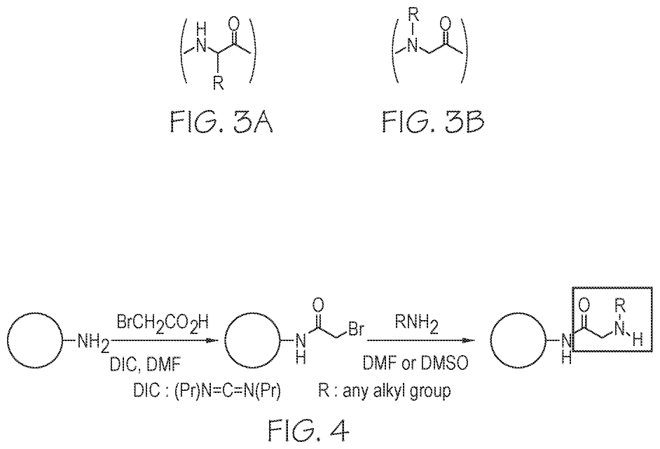

Peptoids (oligo-N-substituted glycines) (FIG. 3B) closely resemble peptides (FIG. 3A) except that their side chains extend from the main chain nitrogen rather than from the α-carbon. These oligomers are achiral, protease-resistant, and highly tissue-permeable.

Moreover, as illustrated in FIG. 4, the synthesis of peptoids is straightforward. In particular, adding one residue (equivalent to a peptide amino acid) requires only two chemical steps, and each of these can be completed by two, 15-second microwave pulses (FIG. 4).

Applicant and others have demonstrated that peptoids are rich sources of protein-binding ligands and are non-immunogenic in mice. Furthermore, Applicant and others have used peptoids for specific delivery of 1,4,7,10-tetraazacyclododecane-1,4,7,10-tetraacetic acid (DOTA) imaging probes into tumors, as peptoid modifications are straightforward and have moderate clearance. Hence, Applicant believes that peptoid are advantageous in imaging probe delivery as compared to standard ligand types such as peptides, aptamers, antibodies, liposomes and nanoparticles.

### Example 1.2. One Bead Two Color (OBTC) Cells Screening and Hit Identification

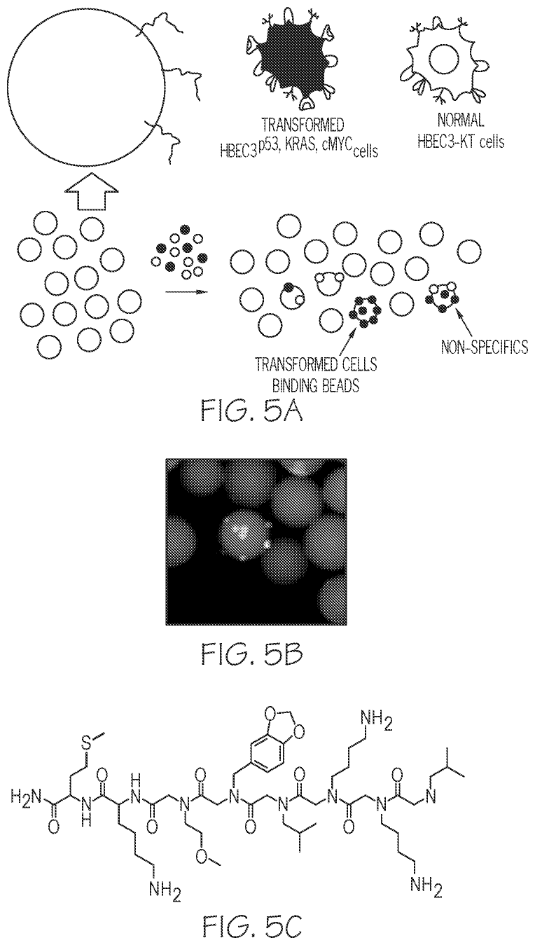

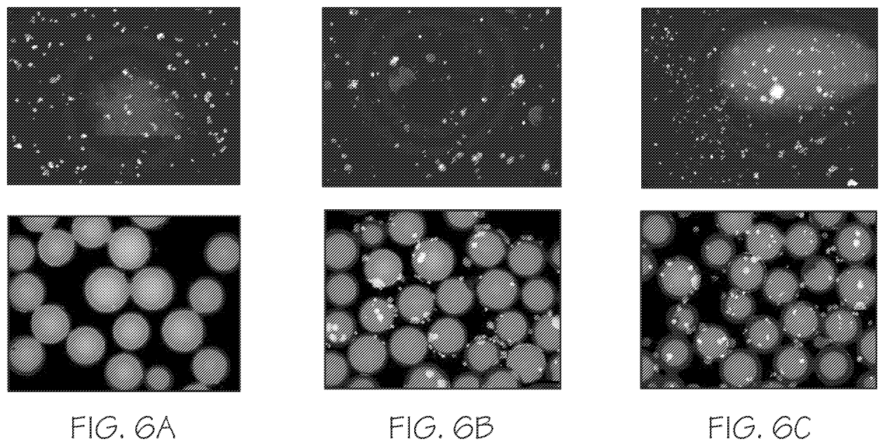

Applicant had previously developed on bead two color (OBTC) combinatorial cell-screening technology that can uniquely recognize differences between two cell surfaces (FIGS. 5A-C). Udugamasooriya et al. A Peptoid “Antibody Surrogate” That Antagonizes VEGF-VEGF Receptor 2 Interaction, J. Amer. Chem. Soc., 2008, 130, 5744-5752. Matharage, J. M. et al. Unbiased Selection of Peptide-Peptoid Hybrids Specific for Lung Cancer Compared to Normal Lung Epithelial Cells, ACS Chem. Biol., 2015, 10 (12), 2891-9. Raymond, A. C., et al. Unbiased peptoid combinatorial cell screen identifies plectin protein as a potential biomarker for lung cancer stem cells, Scientific Reports, 2019 (9), 14954. This assay is based on exposing two identical cell populations, which differ only in the presence (red/dark stained) or absence (green/light stained) of a particular receptor, to a peptoid library carried on beads (one-compound-multi copies per bead). If a bead binds only to cells stained red (i.e., dark), this indicates that the peptoid on this bead binds only to that overexpressed receptor/biomarker and not to any other common cell-surface molecules. If a bead binds to any other cell surface-molecule, it should register green (i.e., light) cells as well and can be discarded at the initial screen level.

FIG. 5A provides a pictorial representation of the OBTC assay. The fully transformed HBEC3^{p53,KRAS,cMYC} cells (FIG. 2, step 4) were stained using red Q-dots and normal cells HBEC3-KT were stained in green using green Q-dots. One million cells of each color were mixed in 1:1 ratio and were incubated for 1 hour with beads (100,000 beads library) containing one-bead one-compound library at 23° C. (FIG. 2A). Applicant identified one red cells bound bead potential hit, which is binding to a receptor present only on transformed cells (HBEC3^{p53,KRAS,cMYC}) but absent in normal cell (HBEC3-KT). FIG. 5C shows the chemical structure of the peptoid JM3A that binds to transformed cells.

### Example 1.3. Initial Confirmation of JM3A Binding Specificity to HBEC3^{p53,KRAS,cMYC} and not to Normal HBEC3-KT Cells

The compound JM3A was resynthesized on tentagel beads and equilibrated with HBEC3^{p53,KRAS,cMYC} (red stained) cells and HBEC3-KT (green stained) cells separately and also as a mixture. As shown in FIGS. 6A-6C, the beads bound only to red stained cells (HBEC3^{p53, KRAS,cMYC}) and not to green stained cells (HBEC3-KT).

### Example 1.4. Target Identification of JM3A as Vimentin Protein on HBEC3^{p53,KRAS,cMYC} Cells

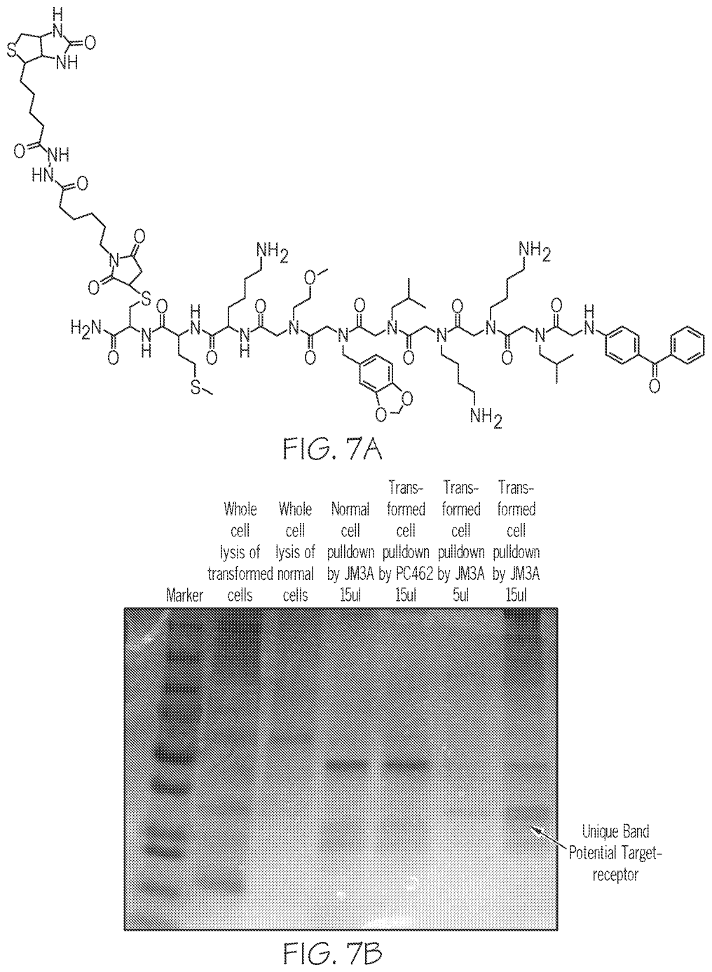

For pulldown assay, the JM3A compound was synthesized with Biotin on C-terminal and benzophenone on its N-terminal (FIG. 7A). The compound was first mounted on streptavidin magnetic beads, followed by incubation of the cells HBEC3^{p53,KRAS,cMYC} and HBEC3-KT for 1 hour. Bound cells were separated using magnetic beads and cross-linked using UV. The cells were then lysed, and the proteins were separated by electrophoresis and visualized by silver staining (FIG. 7B). The unique band at around 60 kDa in the JM3A-bound sample, which was not present in whole cell lysis of HBEC3-KT cells or in the Control-PC462 compound pulldown, was analyzed by standard proteomics technique. The proteomic data suggested vimentin as the target receptor present in HBEC3^{p53,KRAS,cMYC} cells but absent from HBEC3-KT cells.

Vimentin is a 57-kDa protein that is universally expressed in mesenchymal cells. Vimentin belongs to intermediate filament (IF) proteins and used as a marker for epithelial-to-mesenchymal transition (EMT) for normal development and metastatic initiation. A broad range of cell lines expresses vimentin, including cells like neuronal renal tubular cells, endothelial cells lining blood vessels, renal tubular cells, macrophages, neutrophils, fibroblasts, and leukocytes. Vimentin exhibits imperative roles, which include cell adhesion, migration, and signaling. Vimentin constitutively possesses a central α-helical domain, capped by non-α-helical N (head)- and C (tail)-terminal end domains. Two monomers together are associated in parallel formation to form a coiled-coil dimer. Moreover, vimentin is known to form a homopolymer and a heteropolymer.

### Example 1.5. Vimentin as an Early Biomarker for Cancer Detection

Emerging tissue/cytological biomarkers include DNA methylation, miRNA, proteomics, metabolomics, and microbiomics. Additionally, new biomarkers have been described from liquid biopsies, such as circulating tumor cells, exosomes, and plasma miRNA/DNA, including vimentin (the target identified here), reported as a marker in blood and urine.

Applicant and others have shown that vimentin is expressed in precancerous cells. Interestingly, normal cells around precancerous cells battle with through a phenomenon called epithelial defense against cancer (EDAC).

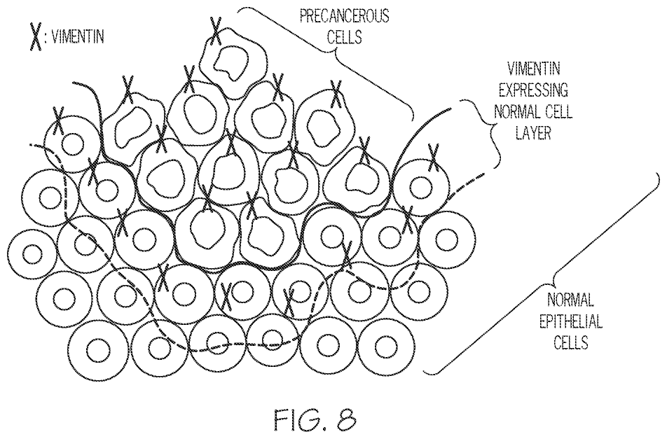

It has been shown that vimentin is overexpressed in those normal epithelial cells in contact with precancerous cells involved in EDAC (FIG. 8—cells between two lines). Thus, vimentin is highly enriched in both active tumor-forming lung nodules and in those cells at the margins. Therefore, Applicant hypothesizes that ⁶⁴Cu-NOTA conjugated vimentin-targeted JM3A has a higher chance of being accumulated in earliest active lung nodules and display a much more sensitive signal in PET-CT. This tumor detection technique will reduce the overwhelming false positives observed in LDCT.

### Example 1.6. Confirmation of JM3A Binding to Vimentin Through on Bead Protein Assay

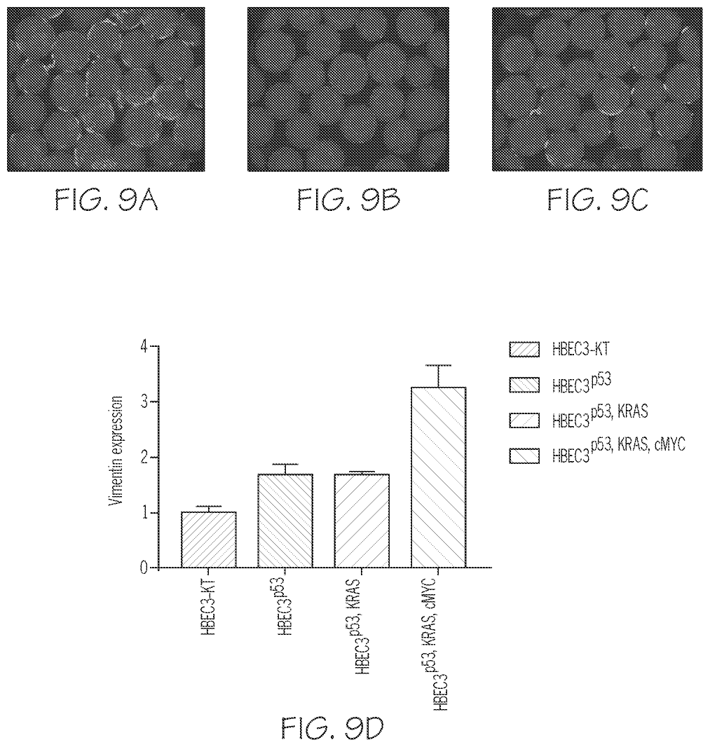

To reconfirm the JM3A-vimentin interaction, an on bead protein binding assay was performed. Vimentin protein (His-tag) was screened with compound JM3A (on tentagel beads) and visualized using a secondary antibody (Anti-His Alexa 647) (FIG. 9A). Nonspecific interactions were evaluated by incubating anti-His Alexa 647 with the beads directly, which did not show any fluorescent signal (FIG. 9B). Further, the JM3A (bead displayed) bound vimentin was competed with free M3A pre-incubation, which reduces the signal indicating JM3A-vimentin specificity (FIG. 9C). Applicant quantified the vimentin expression in the transformed cells using qRT-PCR and the results showed vimentin over-expression in HBEC3^{p53,KRAS,cMYC} in comparison to other cells (FIG. 9D).

### Example 1.7. Confirmation of JM3A Binding to Vimentin Through Western-Blot Assay

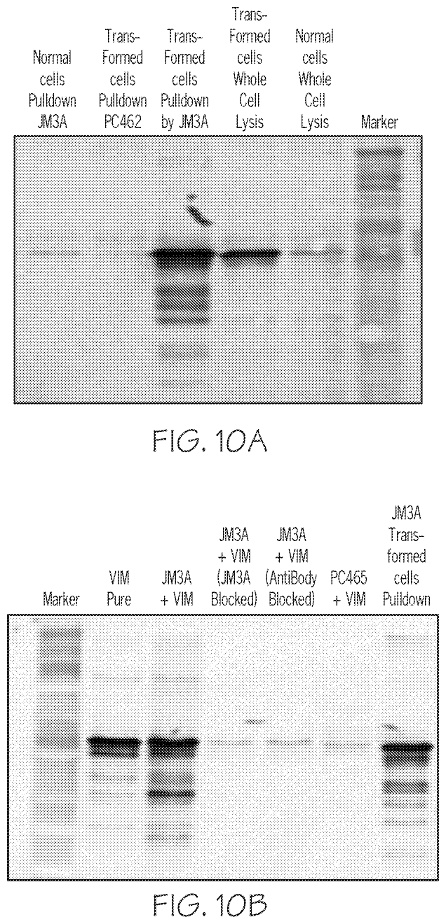

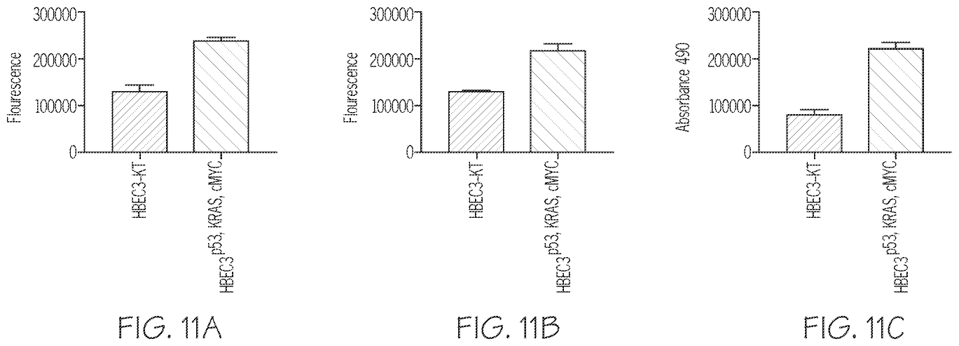

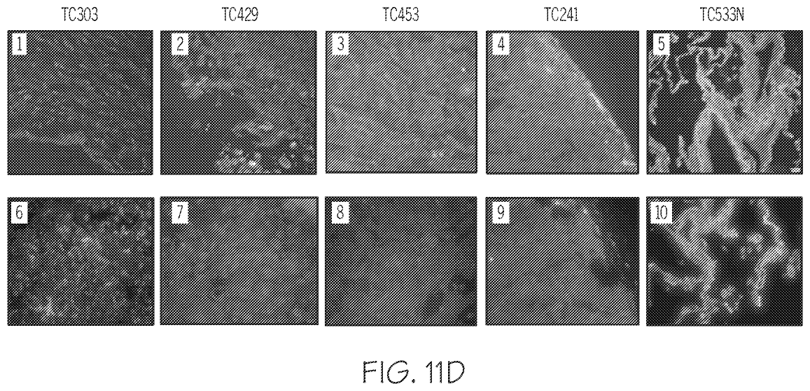

To reconfirm the JM3A-vimentin interaction, western blot analysis of samples from cross-linking and magnetic-bead pulldown assay with normal cells (HBEC3-KT) and transformed cells (HBEC3^{p53,KRAS,cMYC}) were performed (FIG. 10A). Pulldown assay was performed on normal and transformed cells using JM3A and control PC462. All the pulldown fractions were run on western-blot and compared with whole-cell lysis of HBEC3-KT and HBEC3^{p53,KRAS,cMYC} cells. FIG. 10A shows that the vimentin protein was pulldown only with JM3A in transformed cells but not in normal cells. This reconfirmed Applicant's pulldown assay and silver staining experiment shown in FIG. 7B.

To further confirm of JM3A specificity, the western blot assay was repeated using commercially available vimentin protein (FIG. 10B). The compound JM3A showed strong interaction and pulldown of vimentin protein. This JM3A bound band disappeared when this binding was competed (blocked) with 10× free JM3A, and 5× free vimentin antibody. The control compound PC462 did not show any interaction with vimentin. The results were further confirmed by running pure protein as reference band and JM3A transformed cells pulldown proteins.

### Example 1.8. Diagnostic Utility of Peptoid JM3A by Quantification of Cells Overexpressing Vimentin and Detecting Vimentin in Early Cancer Cells and Human Tissues

JM3A can be utilized as an early cancer biomarker to develop simple diagnostic tools such as reading the cells auto fluorescence markers or quantifying the cells using MTS assay. Applicant performed the pulldown assay using JM3A to HBEC3-KT and HBEC3^{p53,KRAS,cMYC} cells. The Biotin-JM3A compound was mounted on streptavidin coated magnetic beads and 1 million cells (HBEC3-KT and HBEC3^{p53,KRAS,cMYC} cells each) were incubated for 1 hour. Next, bound cells were pulled using magnet.

The difference of number of cells pulled down were quantified for NAD (P) H (FIG. 11A) and Dityrosine (FIG. 11B) expression. Also to the pulldown cells MTS reagent was added and number of cells were quantified (FIG. 11C). Using these preliminary assays, Applicant showed that vimentin expressing cells can be pulled down using JM3A and can be quantified using simple and standard tools.

In a more clinically applicable set up, Applicant detected vimentin expression using JM3A in early cancer tissues from human samples using immunohistochemistry (IHC) experiments. Biotin-JM3A was incubated with those tumor tissue cross-sections compared to normal tissues for 1 hour and visualized by streptavidin Q-dots 655 under fluorescent microscope using Long-pass filter (FIG. 11D).

### Example 1.9. Identification of Minimum Pharmacophore of JM3A

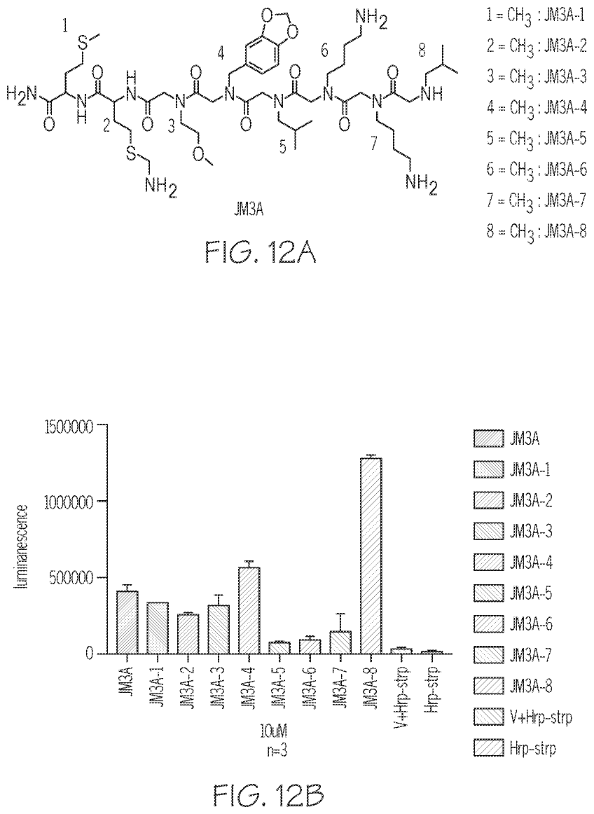

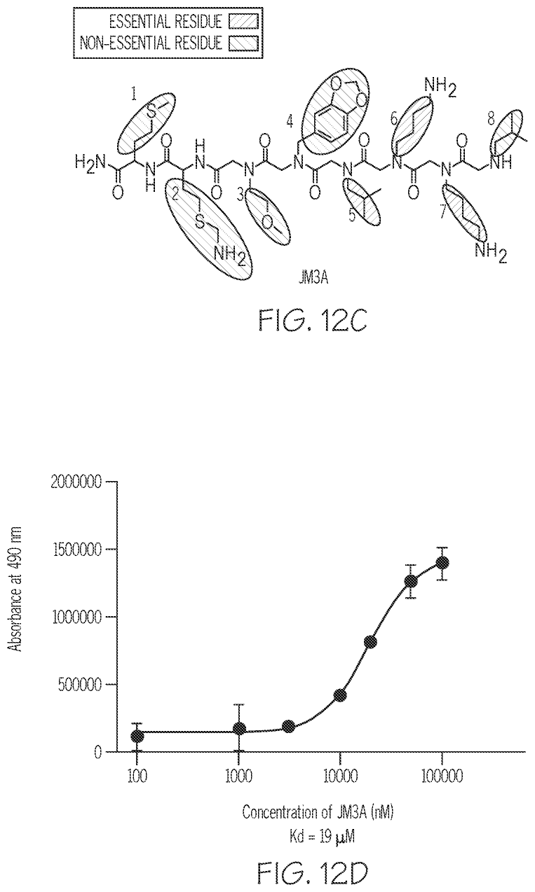

To identify minimum pharmacophore of JM3A (the most important residues directly needed for protein binding), an alanine/sarcosine scan was done. A total of 8 compounds were synthesized replacing 1 residue at a time by alanine (for JM3A-1 and JM3A-2) or sarcosine (for JM3A-3 to JM3A-8) (FIG. 12A). ELISA-like binding assay (FIG. 12B) was performed and Applicant found that residue 5, 6 and 7 are the important residues in JM3A directly needed for vimentin binding (FIG. 12C). The binding quantification indicates that K_{d}=19 μM (FIG. 12D).

### Example 1.10. Effect of New Moiety-Benzophenone at N-Terminal of JM3A

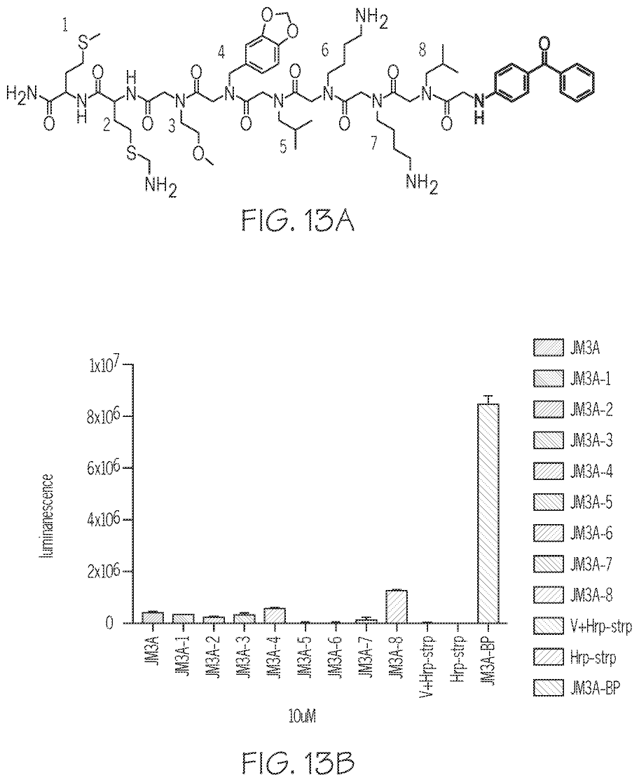

JM3A derivative with benzophenone at its N-terminal (FIG. 13A) was developed for an initial pull-down assay (for target identification). The derivative showed significant improvement in binding of the compound (JM3A-BP) in comparison to all other derivatives in the ELISA-like binding assays (FIG. 13B).

### Example 1.11. Optimization of the JM3A Compound

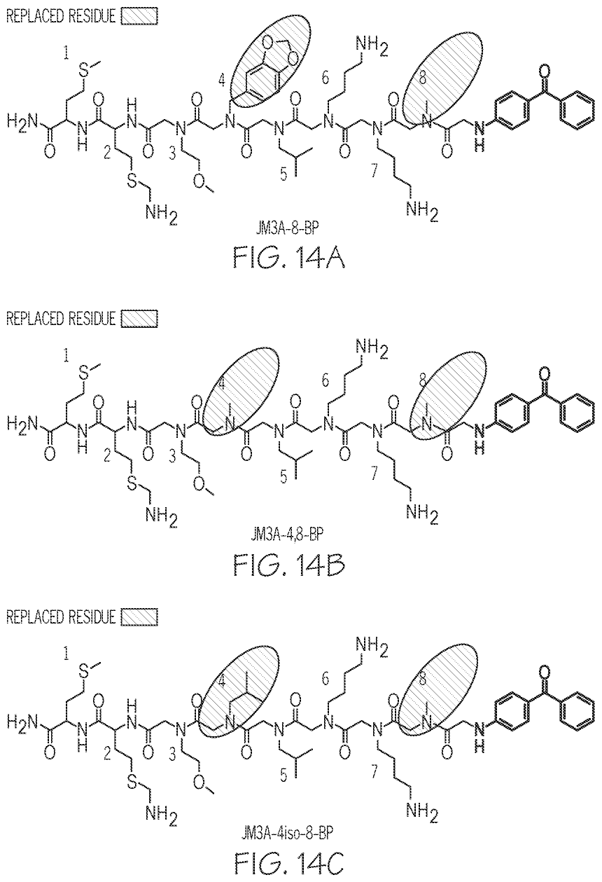

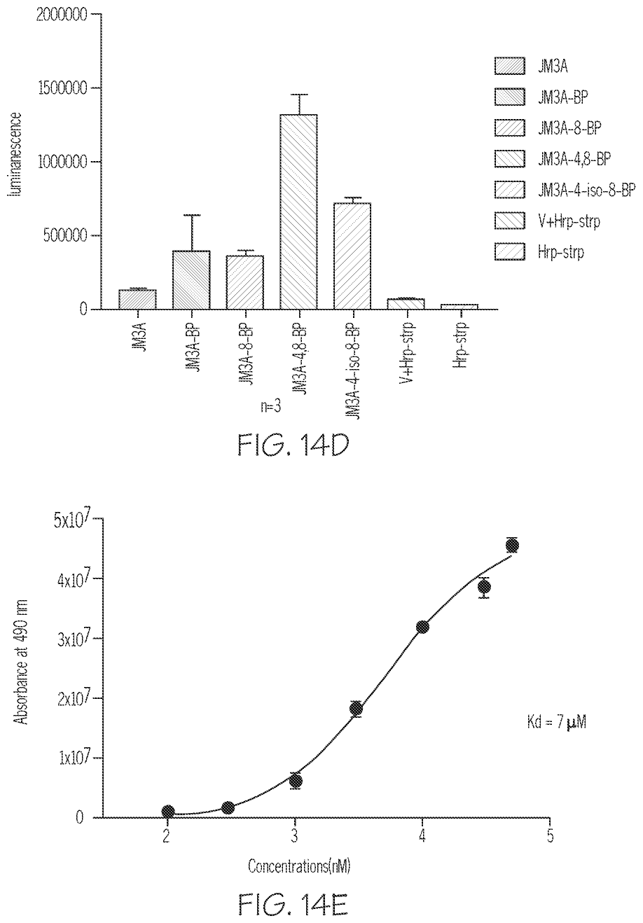

After identifying the minimum pharmacophore of JM3A, and identifying that having benzophenone at the N-terminal significantly improved the binding affinity of the compound, Applicant synthesized three different compounds by changing the 4^{th} and 8^{th} residues of JM3A-BP. Compound JM3A-8-BP has piperonylamine as its fourth residue, compound JM3A-4,8-BP has sarcosine as 4^{th} residue and compound JM3A-4-iso-8-BP has isobutyl amine as its 4^{th} residue (FIGS. 14A-C). Binding affinity of all three compounds to vimentin protein was compared using ELISA-like binding assay (FIG. 14D) and quantification indicates that K_{d}=7 μM for JM3A-4,8-BP (FIG. 14E).

Applicant found that having JM3A-4,8-BP having sarcosine as 4^{th} residue has shown highest binding affinity in comparison to all other derivatives. Binding quantification shows K_{d}=7 μM.

### Example 1.12. Dimerization of JM3A Derivatives to Further Improve Binding

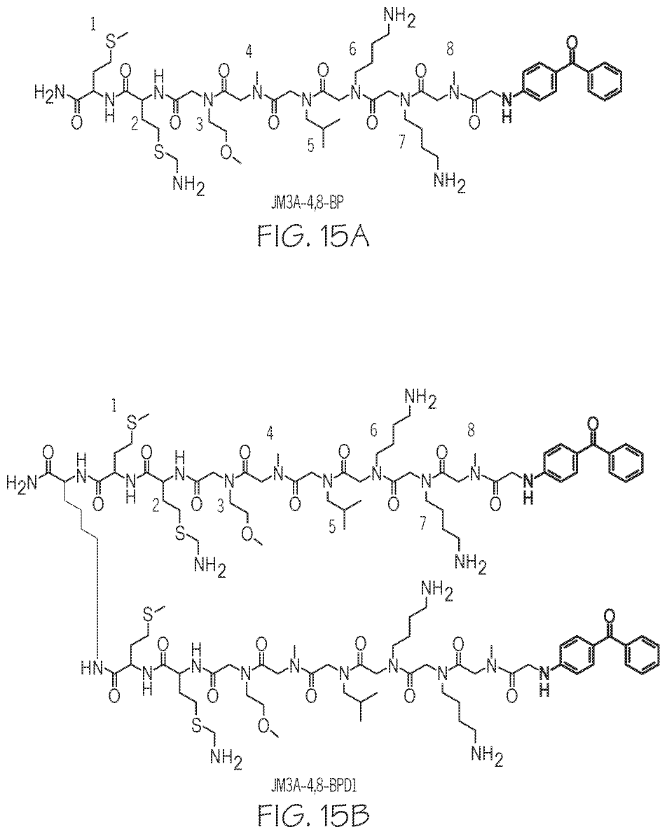

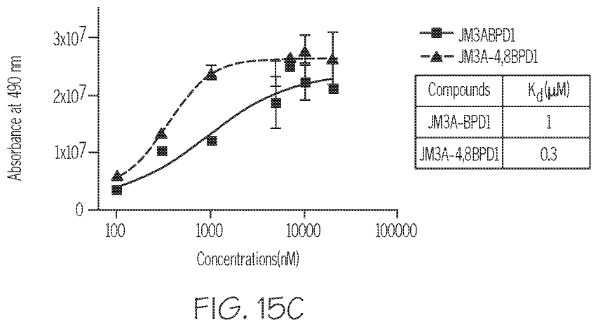

After testing the monomeric derivatives, Applicant further improved the binding affinity of JM3A derivatives by linking the compounds to its C-terminal using Lysine as a linker and making dimer derivatives. Applicant compared the binding affinity of dimer derivatives and found that the compound JM3A-4,8-BPD1 showed stronger binding to the vimentin protein as compared to rest of the derivatives (FIGS. 15A-C), displaying a K_{d} of 300 nM.

### Example 1.13. Optimization of Dimer Derivatives by Replacing Benzophenone to Few Other Bi-Phenyl Derivatives

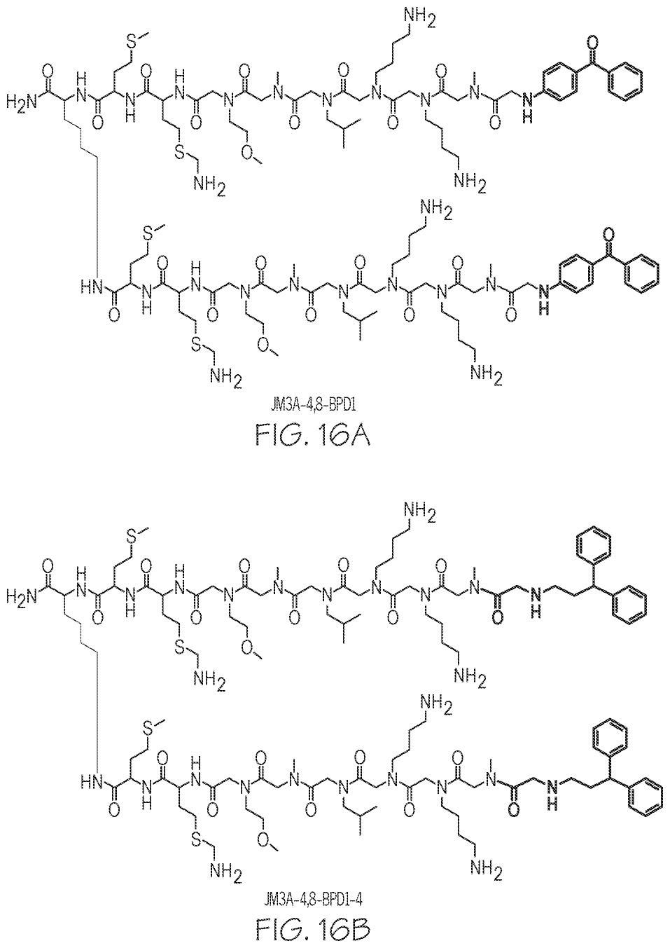

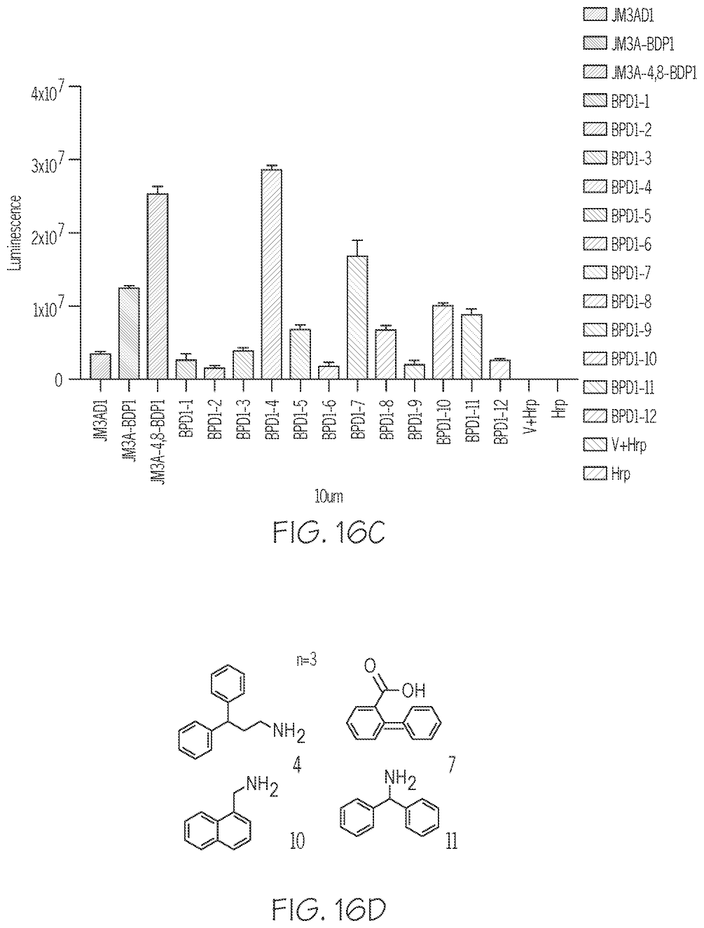

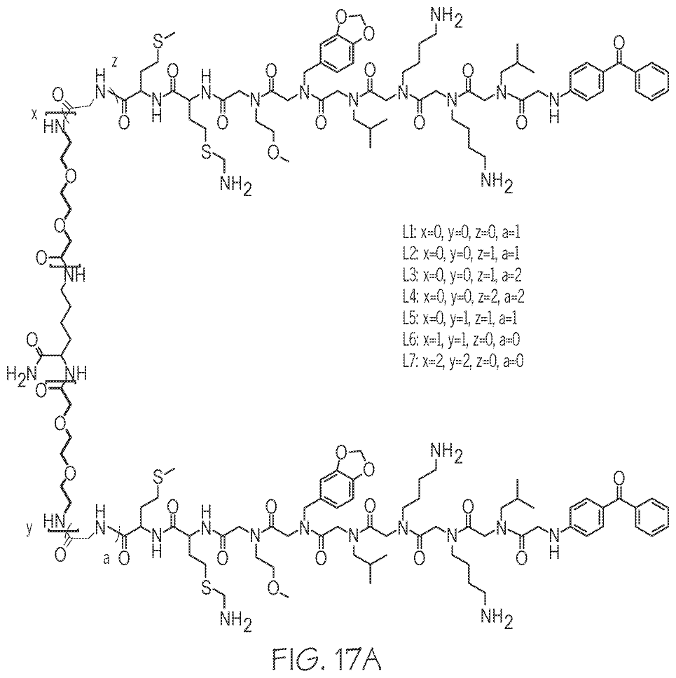

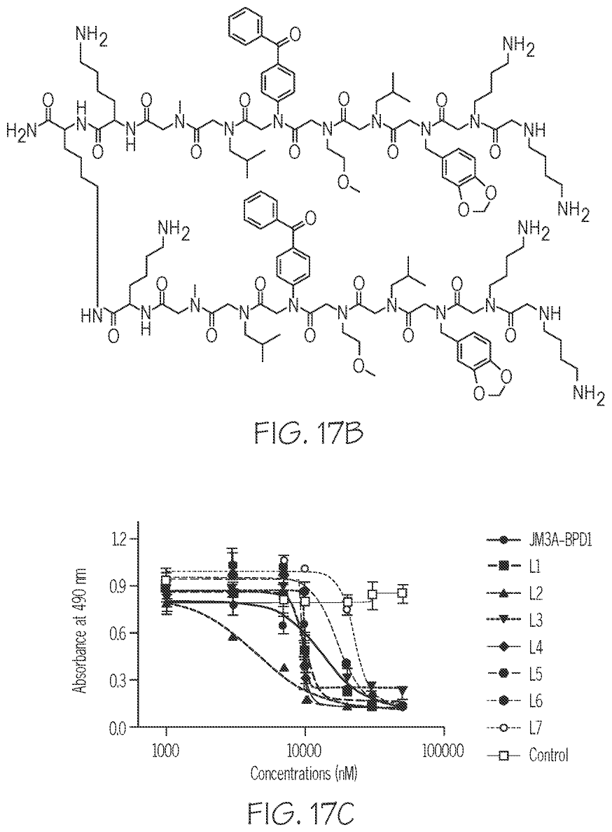

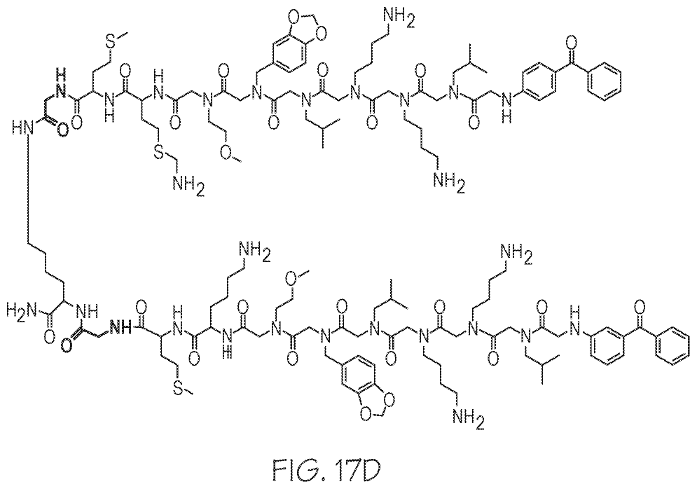

Applicant further synthesized 11 different dimer compounds replacing the benzophenone moiety by biphenyl derivatives that are structurally similar to benzophenone. Applicant compared the newly synthesized compound binding affinity with vimentin using ELISA-like binding assay (FIG. 16C). Applicant found that compound JM3A-4,8-BPD1-4 (FIG. 16B) was having similar binding affinity to JM3A-4,8-BPD1 (FIG. 16A). A total of four (4) derivatives showed significant binding affinity (FIG. 16D).

### Example 1.14. Identification of Tumor-Suppressing JM3A-BPD1 with Optimized Linker Length

Since cell surface receptors are displayed in copies, the multimerization method has been considered an ideal strategy to successfully extract avidity for ligand binding cell surface receptors. Applicant previously reported that dimeric peptoids exhibited enhanced binding activity and biological activity compared to their monomeric versions. The length, geometry, and physiochemical properties of the linker may play an important role in ligand binding cell surface receptors. Accordingly, Applicant focused on studying how various linker lengths between two JM3A-BP monomers could impact their therapeutic effects.

Applicant first connected two JM3A-BP monomers at C-terminus with lysine as a central linker. The Glycine or Polyethylene glycol (PEG) moieties were employed to extend the linker length by coupling to one/two amine functionalities. The synthesis method is the same as Fmoc solid-phase synthesis strategy. A total of 7 JM3A-BPD1 homodimer linker derivatives and a control compound with scrambled residues were synthesized.

Next, Applicant evaluated the anti-proliferative activity of these peptoid dimers via standard MTS assays on non-small cell lung cancer (NSCLC) H1299 cells, which have high vimentin expression. The result suggested that the homodimer peptoids displayed anti-proliferation activity in a dose-dependent manner.

One compound L2 with lysine as a central linker and one glycine at each arm was selected due to the highest activity (IC₅₀˜6 μM). The control compound didn't display activity. Notably, the linker length of homodimer peptoids is shorter or longer than lysine. Additionally, two glycines exhibited a loss of activity.

### Example 1.15. Optimization of L2 by Replacing Methionine with Alanine and D-Alanine

The optimized homodimer L2 exhibited better activity compared to other derivatives. However, the methionine at its C-terminus resulted in a very low yield in the synthesis process since the methionine tends to oxidize.

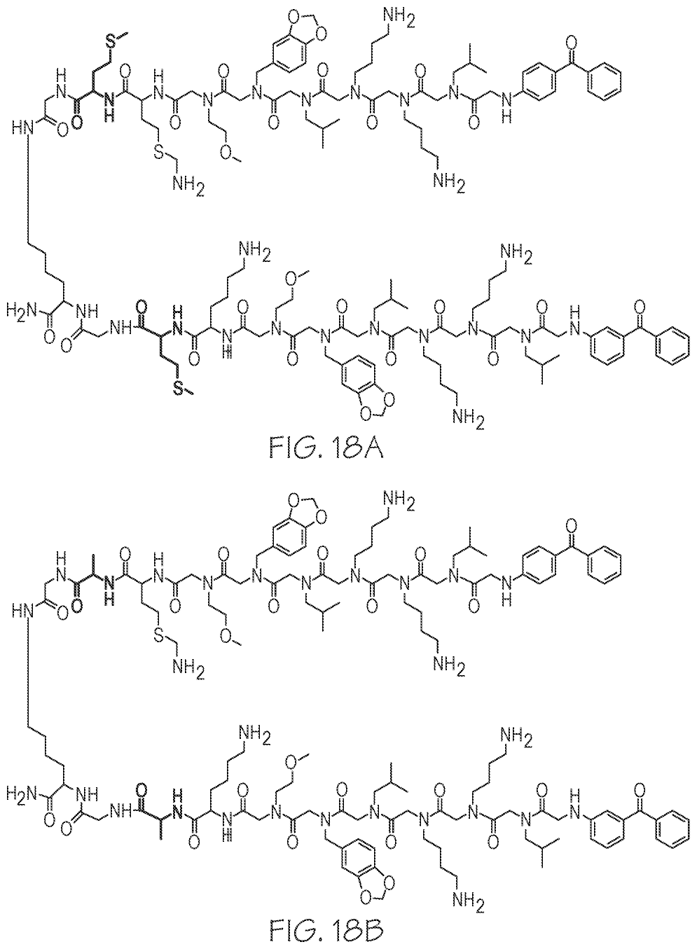

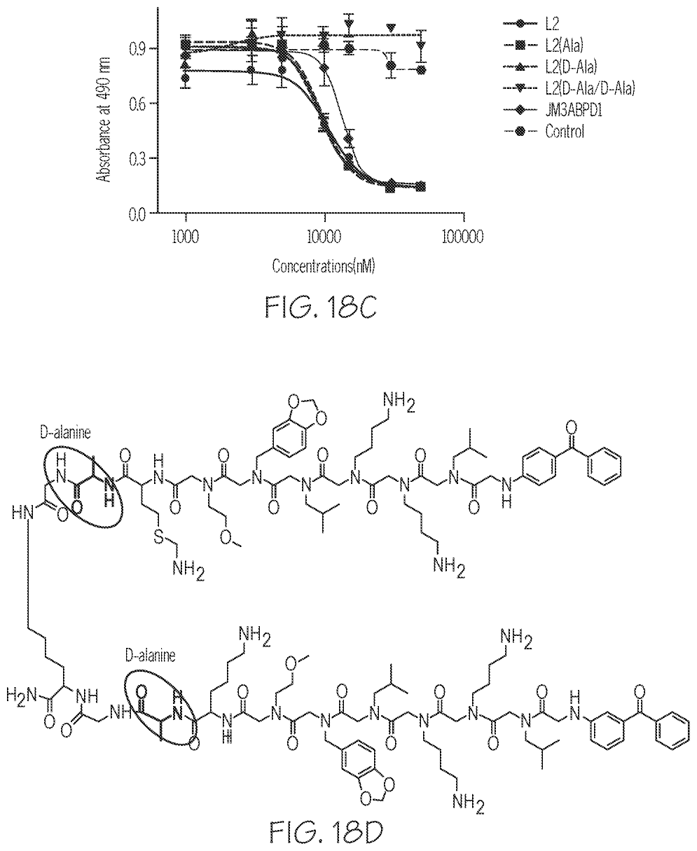

Applicant had previously identified methionine as a non-essential residue in JM3A's binding vimentin. Accordingly, Applicant replaced methionine with alanine, and the yield improved 15 times. Since both glycine and alanine at the C-terminus of L2 are non-essential amino acids that may undergo cleavage in in vivo studies, Applicant replaced glycine and alanine with D-alanine, respectively. The standard MTS assay was used to evaluate the anti-proliferation activity of these homodimer peptoids. The result suggested that L2, L2 (Ala), and L2 (D-ala) exhibited a very similar activity. Applicant unexpectedly observed that, when both glycine and alanine were replaced with D-alanine, the activity was completely abolished. This observation indicated that the spatial arrangement of each JM3A-BP monomer plays an important role in L2 binding vimentin. The results and structures are summarized in FIGS. 18A-D.

### Example 1.16. L2 (D-Ala) Exhibited Improved Binding Activity

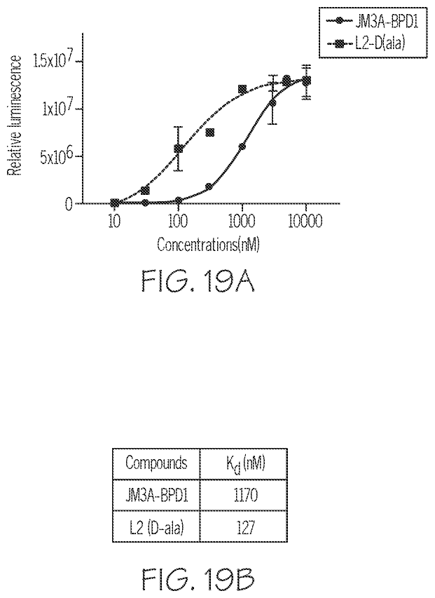

Next, Applicant compared binding activity between L2 (D-ala) and JM3ABPD1 by employing ELISA-like binding assay in a concentration between 10 nM to 10 μM. The data suggested the dissociation concentration (K_{d}) value of L2 (D-ala) is about 127 nM, which is approximately 10 times better than JM3A-BPD1. The results are summarized in FIGS. 19A-B.

### Example 1.17. L2 (D-Ala) Disrupts Cell Proliferation on Various Non-Small Cell Lung Cancer Cells

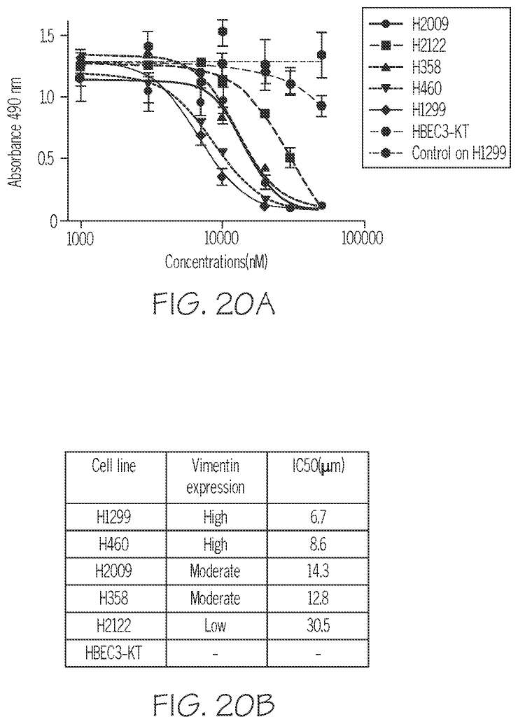

In order to investigate the broader applicability of vimentin targeting in NSCLC cells, Applicant performed the same MTS studies on different lung cancer models with various vimentin expressions. These cell lines included H460 (Carcinoma, large cell lung cancer, male origin), H2009 (Adenocarcinoma, Stage 4, female origin), H358 (Bronchioalveolar Carcinoma, NSCLC, male origin), H2122 (Adenocarcinoma, NSCLC, female origin), and normal bronchial epithelial HBEC3KT cells. The MTS data indicated that L2 (D-ala) exhibited the best activity on vimentin high expression H1299 and H460 cells. L2 (D-ala) displayed medium activity and lowest activity on vimentin moderate expression cells H2009, H358, and vimentin low expression H2122 cells respectively. Unsurprisingly, the L2 (D-ala) exhibited inactivity on normal bronchial epithelial HBECK-3KT cells. The results are summarized in FIGS. 20A-B.

### Example 1.18. L2 (D-Ala) Disrupts Cell Proliferation Starting from Two Hours

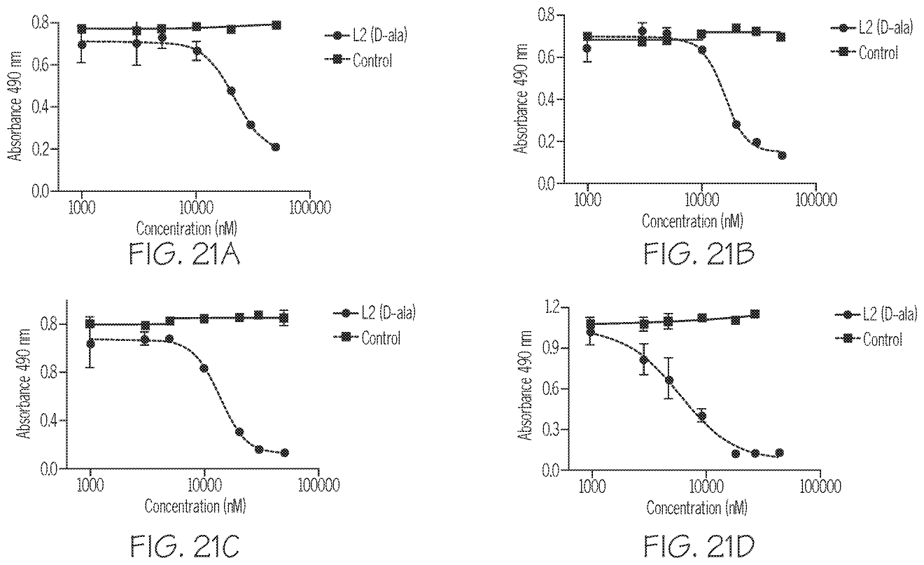

Next, Applicant measured the anti-proliferation capacity of L2 (D-ala) on H1299 cells in a time-coursed manner as shown in FIGS. 21A-D. The Data unraveled that L2 (D-ala) exhibited activity after 2 hours of treatment (FIG. 21A) with an IC₅₀ value of about 21 μM. With the increase in the time of treatment to 6 hours, the L2 (D-ala) displayed an improvement in potency (FIG. 21B). After 18 hours, the activity reached the maximum (IC₅₀˜6 μM) (FIG. 21C). The extension of treatment time after 18 hours had no significant impact on the cells' proliferation (FIG. 21D).

### Example 1.19. L2 (D-Ala) Inhibits Cell Migration Activity

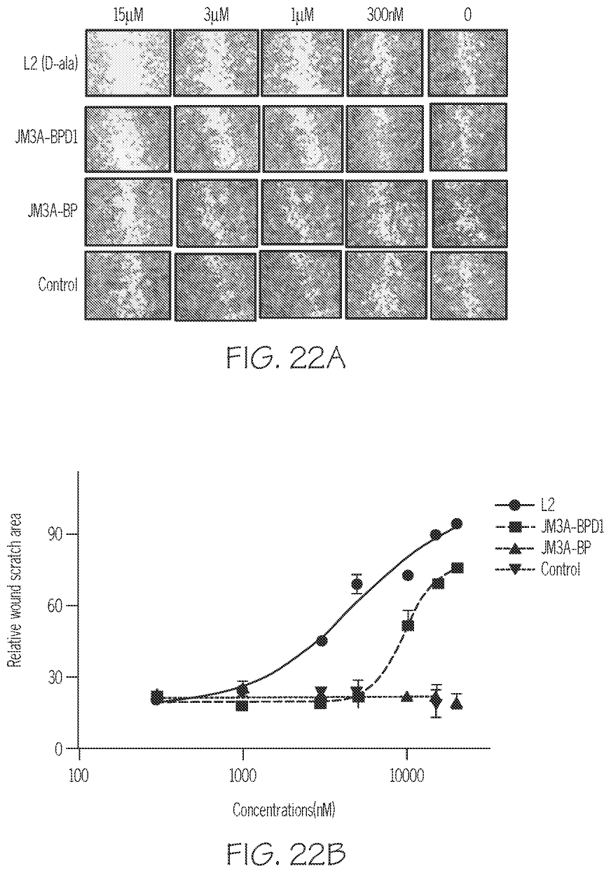

Since vimentin has been identified as an important biomarker of mesenchymal cells during the epithelial-mesenchymal transition (EMT) process. The EMT process can endow epithelial cells with stemness as well as invasion and migration properties. Therefore, Applicant would like to investigate how L2 (D-ala) targeting vimentin can impact cancer stem cells activity. The wound-healing assay indicates the mobility/motility of cells, which is a hallmark measurement of cancer stem cells. The L2 (D-ala), JM3A-BPD1, JM3A-BP, and control compound were used to study the effects on cell migration via wound healing assay. As shown in FIGS. 22A-B, the results suggest that the L2 (D-ala) can inhibit cell migration starting from 3 μM and significantly inhibit the process at 15 μM. The JM3A-BP dimer can moderately inhibit cell migration at 15 μM. Both JM3A-BP monomer and control compounds had no effect on cell migration.

### Example 1.20. L2 (D-Ala) Disrupts In Vitro Colony Formation

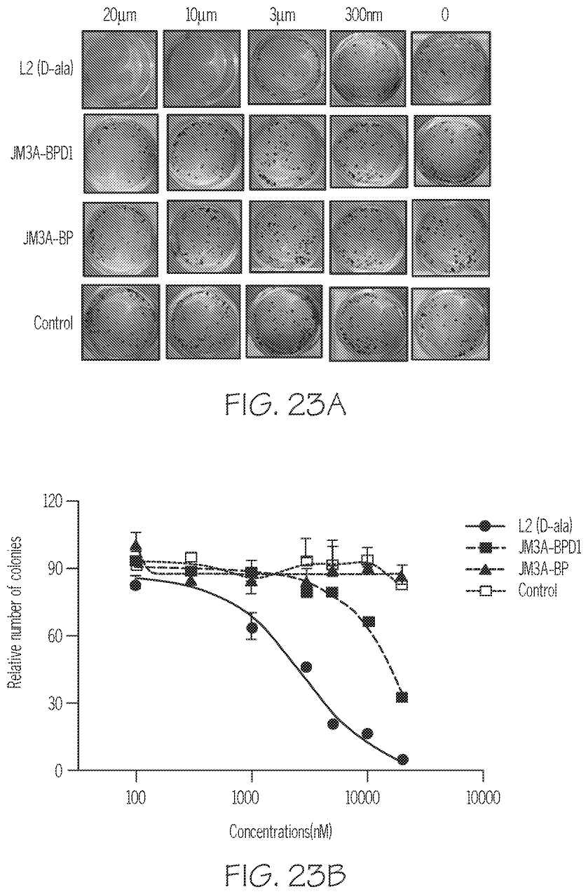

Colony formation is a standard assay to assess the activity of cancer stem cells. When cancer cells are seeded in a low number, normal cancer cells die due to the loss of cell-cell communications while cancer stem cells can survive and form colonies in isolation. Applicant performed colony formation assay on H460 cells to study how L2 (D-ala) can affect cancer stem cells activity. The H460 cells were seeded and treated with L2 (D-ala), JM3A-BPD1, JM3A-BP, and control compounds from day 1. After 7 days of incubation, colonies were carefully counted and plotted. As shown in FIGS. 23A-B, the data suggested that L2 (D-ala) can disrupt colony formation starting from 1 μM and inhibit all colonies at 20 μM. JM3A-BPD1 had some colony disruption at 20 μM. Both JM3A-BP and the control compound displayed activity.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A peptoid selected from the group consisting of: 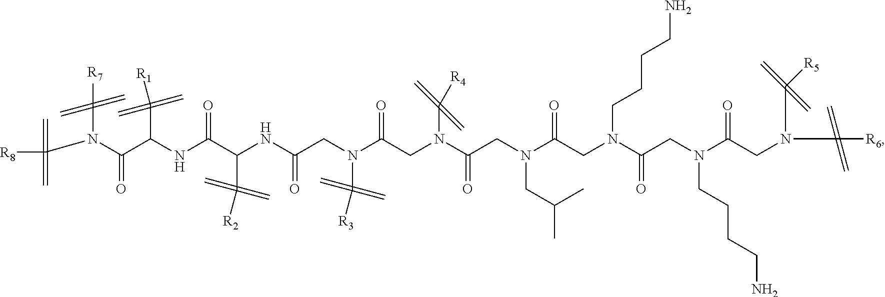
a multimer thereof, a derivative thereof, and combinations thereof,
wherein R₁, R₂, R₃, R₄, R₅, R₆, R₇, and R₈ (R groups) are each independently selected from the group consisting of 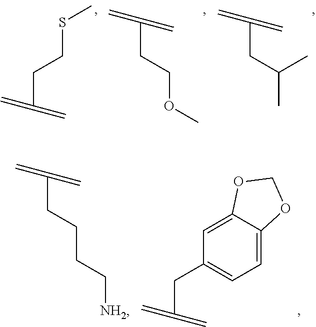
alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof.

2. The peptoid of claim 1, wherein the peptoid is selected from the group consisting of 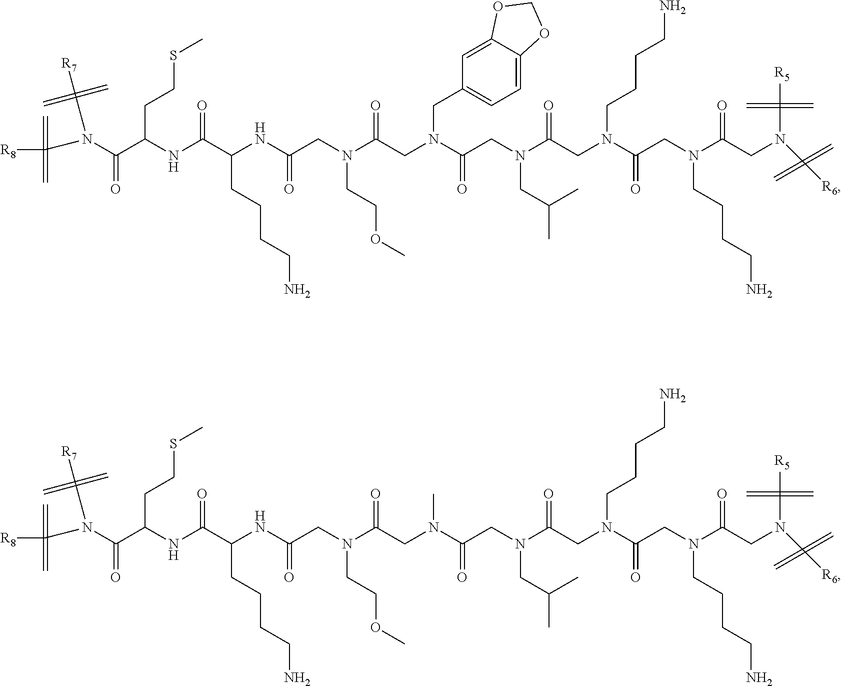 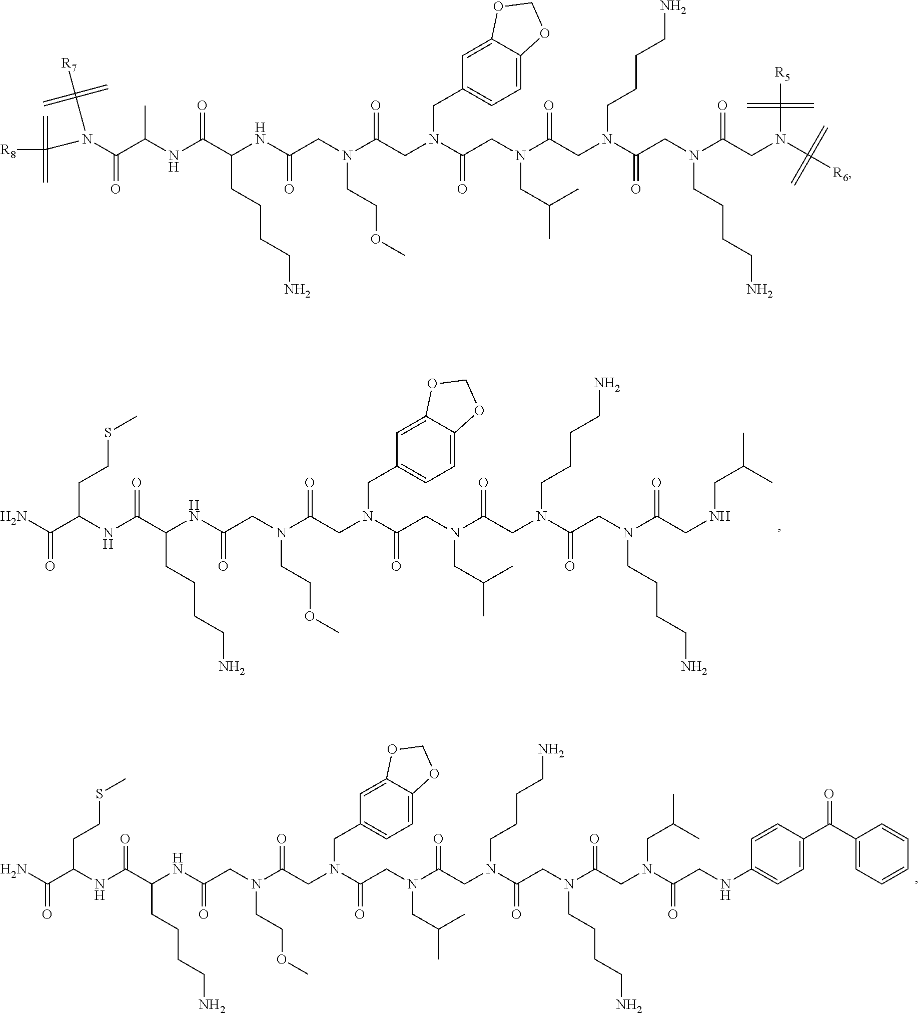 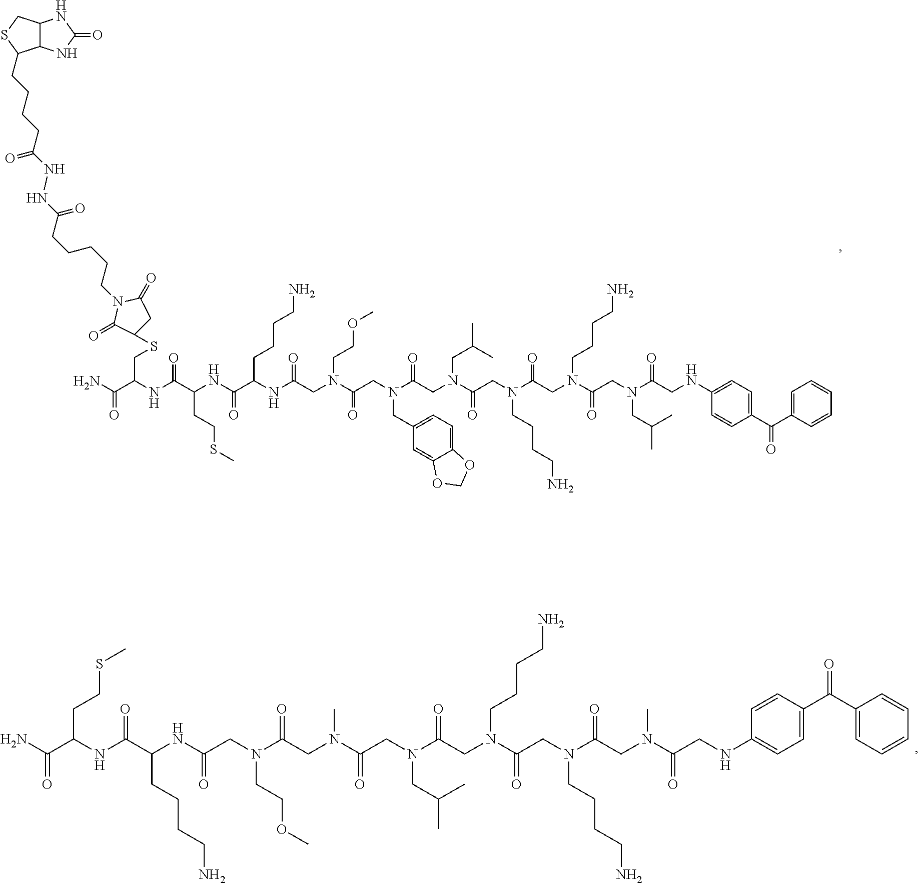 [SMILES: COCCN(CC(=O)NC(CCCCN)C(=O)NC(CCSC)C(=O)NC(CSC1CC(=O)N(CCCCCC(=O)NNC(=O)CCCCC2SCC3NC(=O)NC32)C1=O)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1 ; COCCN(CC(=O)NC(CCCCN)C(=O)NC(CCSC)C(N)=O)C(=O)CN(C)C(=O)CN(CC(C)C)C(=O)CN(CCCCN)C(=O)CN(CCCCN)C(=O)CN(C)C(=O)CNc1ccc(C(=O)c2ccccc2)cc1]
multimers thereof, derivatives thereof, and combinations thereof.

3. (canceled)

4. The peptoid of claim 1, wherein the peptoid is in the form of a monomer.

5. The peptoid of claim 1, wherein the peptoid is in the form of a multimer, wherein peptoids in the multimer are connected through one or more covalent linkages on peptoid backbones, R groups, and combinations thereof; and wherein the covalent linkages are positioned at the C-terminus of peptoids, the N-terminus of peptoids, regions proximal to the N-terminus of peptoids, middle regions of peptoids, regions proximal to the C-terminus of peptoids, and combinations thereof.

6. (canceled)

7. The peptoid of claim 5, wherein the covalent linkages comprise the R₇ group, the R₈ group, one or more linkers, and combinations thereof.

8-9. (canceled)

10. The peptoid of claim 5, wherein the multimer is selected from the group consisting of: 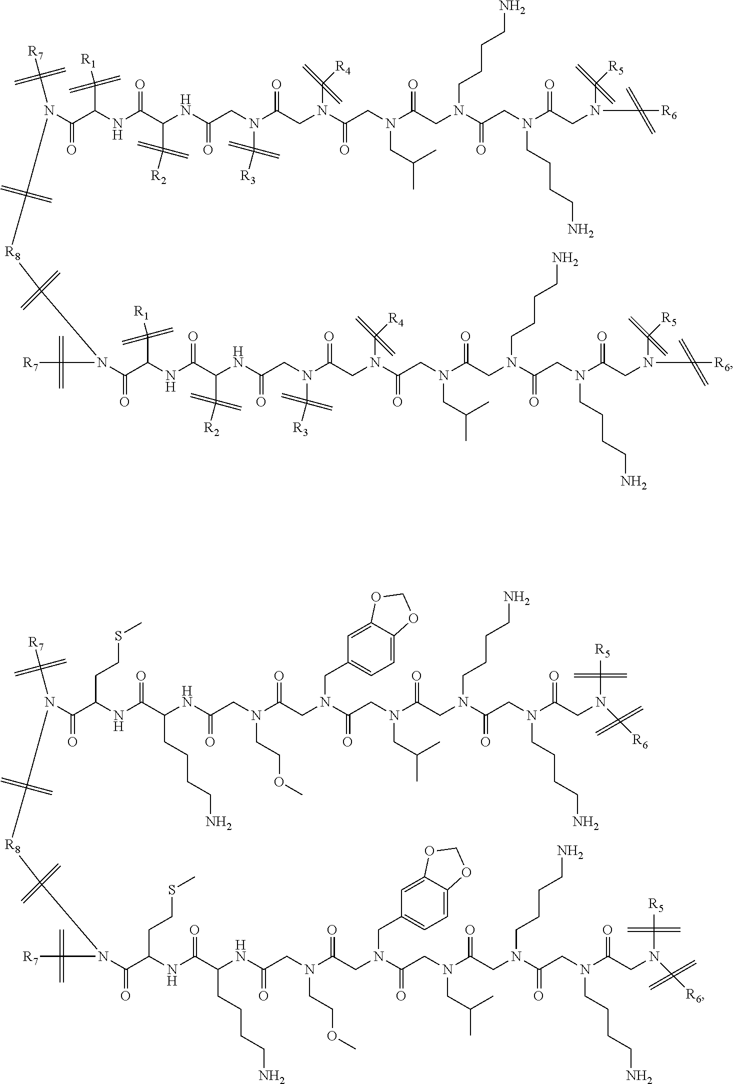 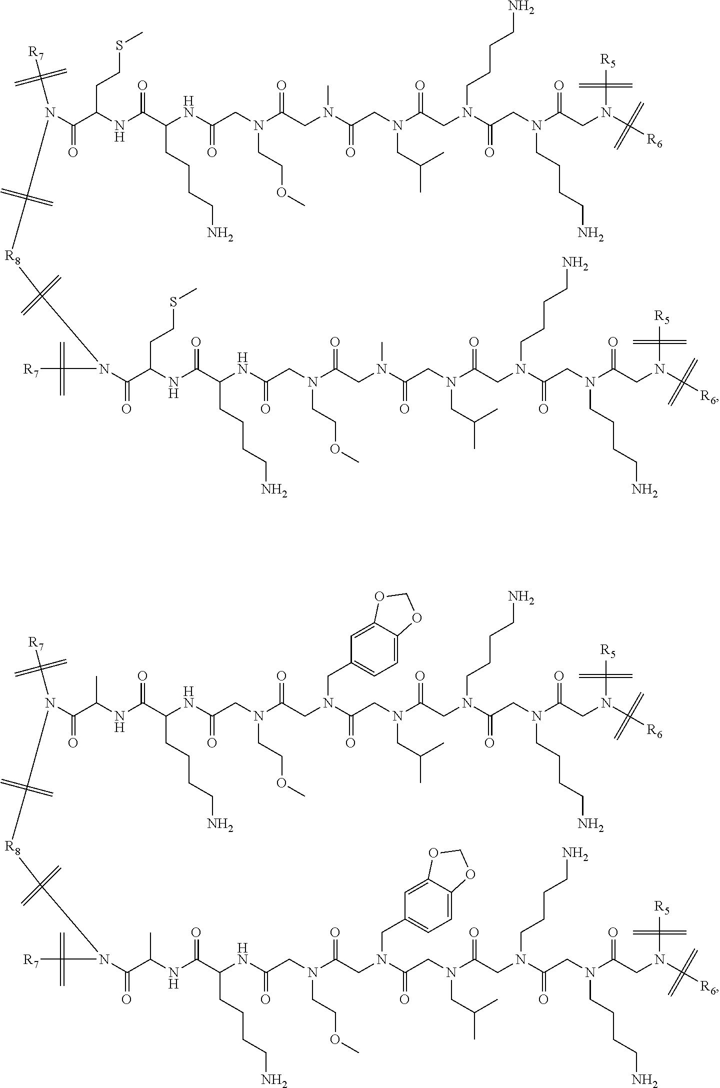
derivatives thereof, and combinations thereof.

11. The peptoid of claim 5, wherein the peptoid is selected from the group consisting of: 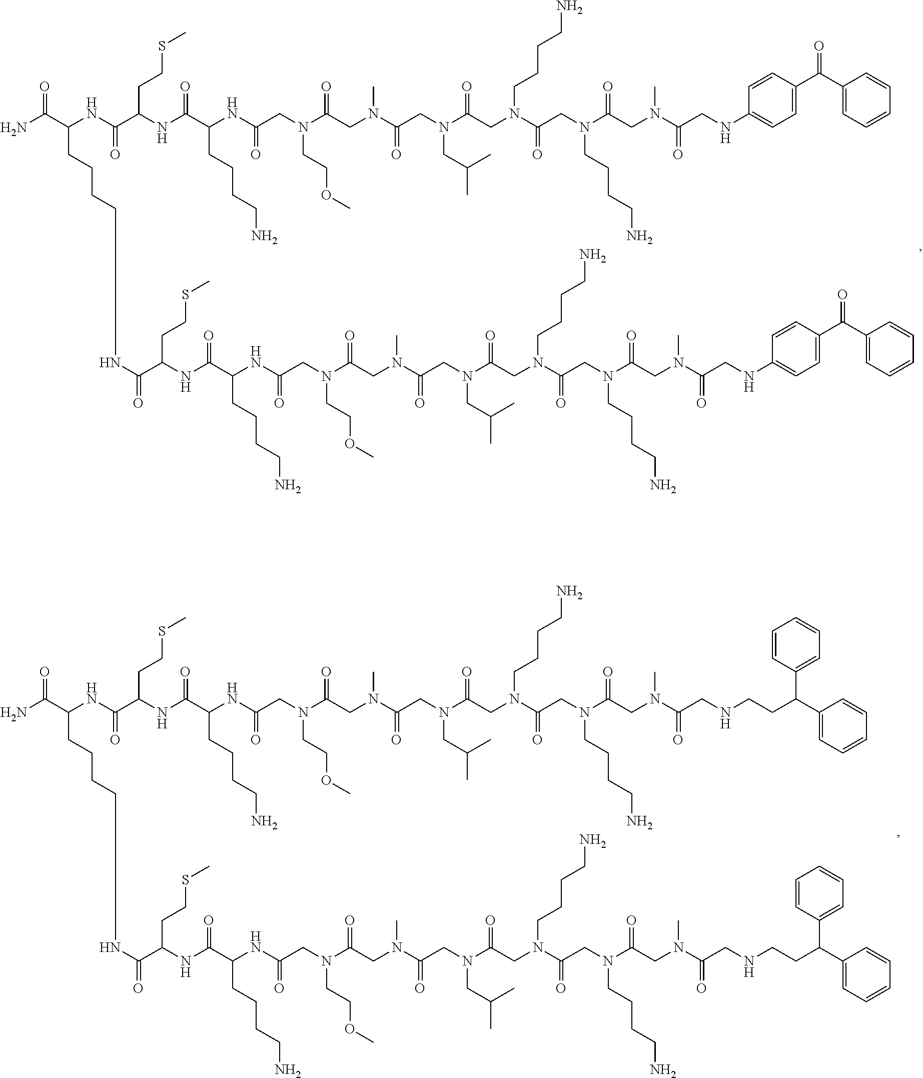 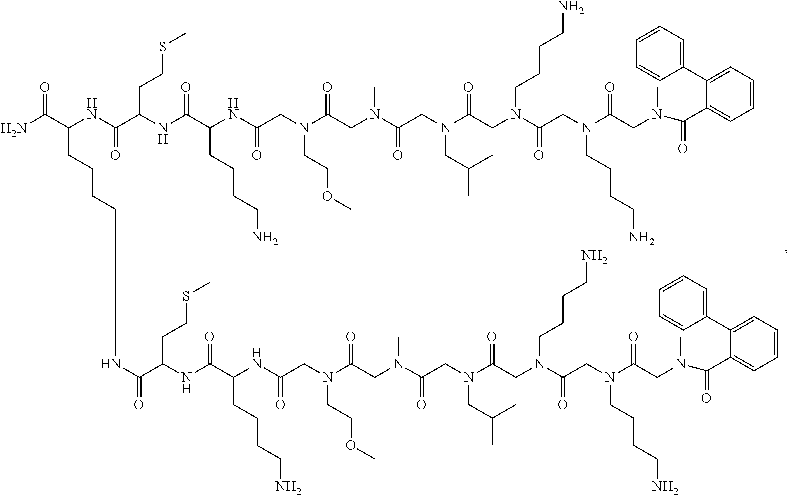 [SMILES: COCCN(CC(=O)NC(CCCCN)C(=O)NC(CCSC)C(=O)NCCCCC(NC(=O)C(CCSC)NC(=O)C(CCCCN)NC(=O)CN(CCOC)C(=O)CN(C)C(=O)CN(CC(C)C)C(=O)CN(CCCCN)C(=O)CN(CCCCN)C(=O)CN(C)C(=O)c1ccccc1-c1ccccc1)C(N)=O)C(=O)CN(C)C(=O)CN(CC(C)C)C(=O)CN(CCCCN)C(=O)CN(CCCCN)C(=O)CN(C)C(=O)c1ccccc1-c1ccccc1] 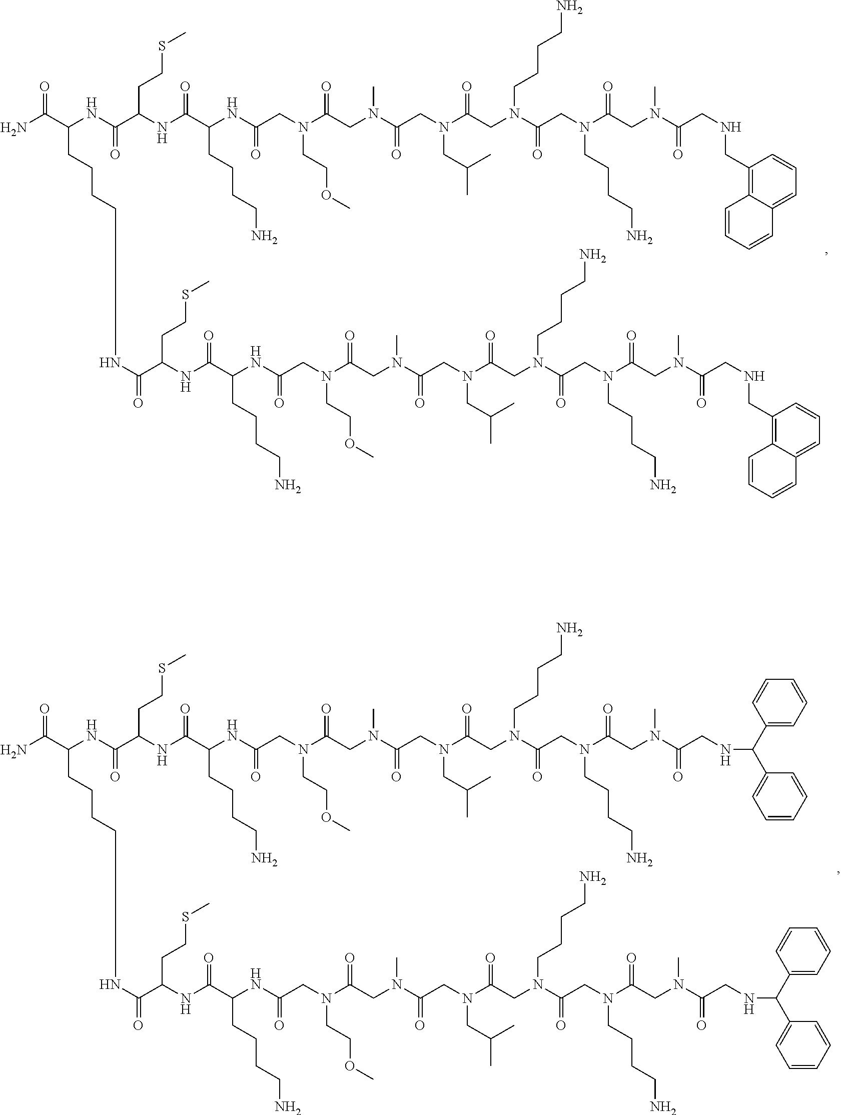 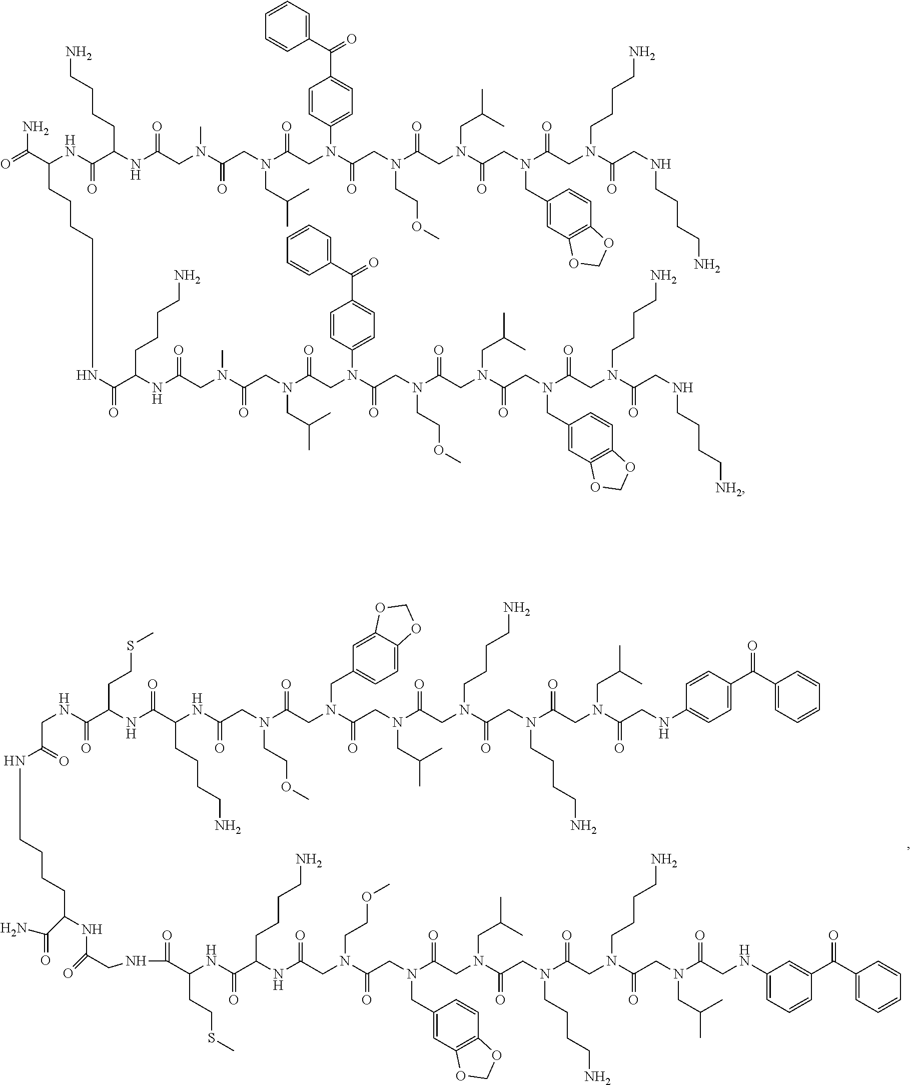 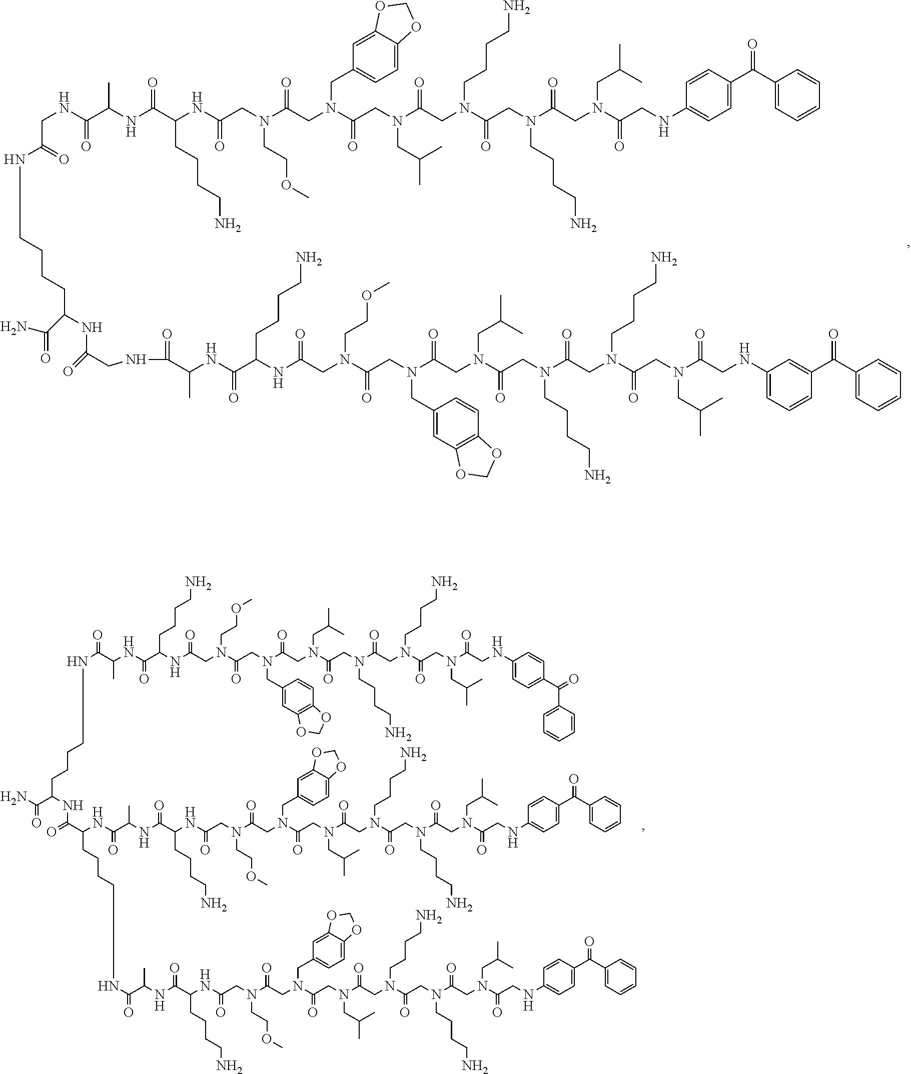 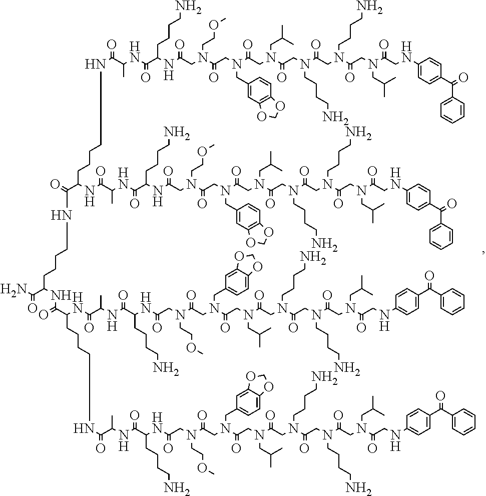
derivatives thereof, and combinations thereof.

12-14. (canceled)

15. The peptoid of claim 1, wherein the peptoid comprises one or more peptoid derivatives, wherein the one or more peptoid derivatives comprise one or more peptoid moieties derivatized with one or more functional groups, and wherein the one or more peptoid moieties are positioned on peptoid backbones, R groups, and combinations thereof, and wherein the one or more functional groups are selected from the group consisting of alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof.

16. (canceled)

17. The peptoid of claim 1, wherein the peptoid is associated with one or more tracing agents selected from the group consisting of fluorophores, chromophores, dyes, radio-labeled molecules, radioactive nuclei, high contrast agents, gadolinium, gallium, thallium, fluorinated compounds, biotin, phenyl-based tracing agents, biphenyl-based tracing agents, benzophenone-based tracing agents, polycyclic aromatic tracing agents, photoaffinity labeling agents, drugs, and combinations thereof.

18. The peptoid of claim 17, wherein the tracing agents are linked to or represented by at least one of the R groups.

19. The peptoid of claim 18, wherein the tracing agents are linked to or represented by the R₅ group, the R₆ group, and combinations thereof.

20-31. (canceled)

32. A method of treating or preventing a cancer in a subject, said method comprising:
administering to the subject a peptoid selected from the group consisting of: 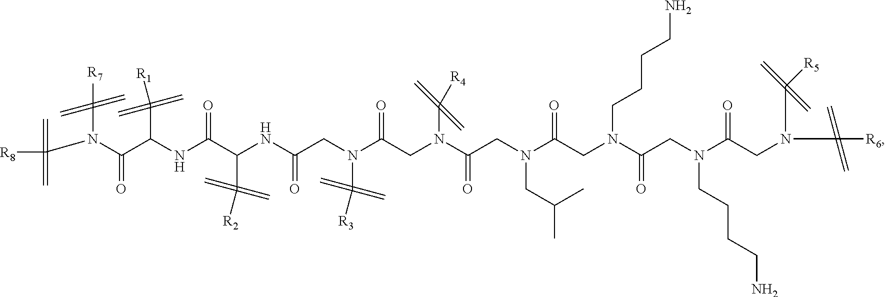
a multimer thereof, a derivative thereof, and combinations thereof,
wherein R₁, R₂, R₃, R₄, R₅, R₆, R₇, and R₈ (R groups) are each independently selected from the group consisting of 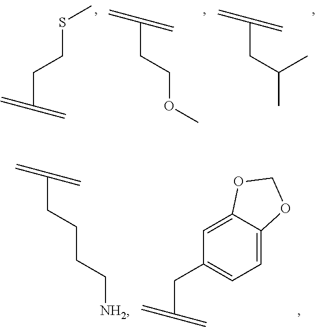
alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof.

33. The method of claim 32, wherein the cancer is selected from the group consisting of lung cancer, non-small cell lung cancer, colon cancer, esophageal cancer, breast cancer, melanoma, prostate cancer, cervical cancer, and combinations thereof.

34. The method of claim 32, wherein the subject is a human being suffering from the cancer.

35. The method of claim 32, wherein the method is used to treat the cancer in the subject.

36. The method of claim 32, wherein the method is used to prevent the cancer in the subject.

37. A method of detecting cancer in a subject, said method comprising:
exposing cells susceptible of being cancerous to a peptoid selected from the group consisting of: 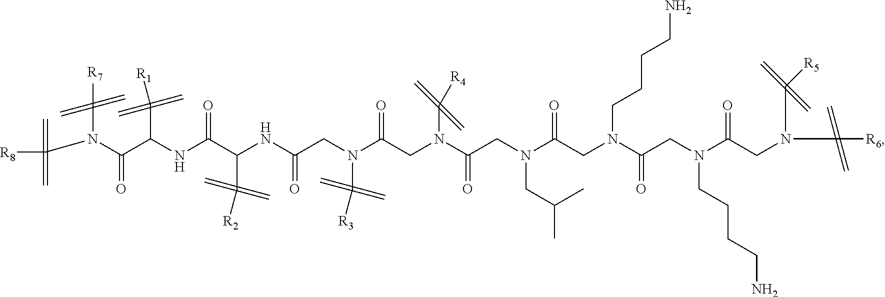
a multimer thereof, a derivative thereof, and combinations thereof,
wherein R₁, R₂, R₃, R₄, R₅, R₆, R₇, and R₈ (R groups) are each independently selected from the group consisting of 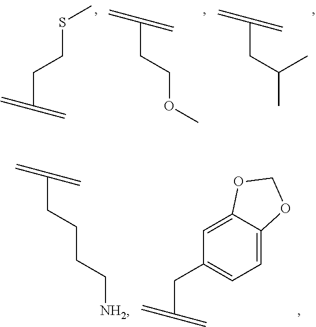
alkanes, alkenes, ethers, alkynes, alkoxyls, aldehydes, carboxyls, hydroxyls, hydrogen, sulfur, phenyls, cyclic rings, aromatic rings, aliphatic rings, heterocyclic rings, linkers, methyl, aliphatic groups, hydrogen groups, amino acid R groups, tracing agents, derivatives thereof, and combinations thereof,
detecting the presence or absence of vimentin associated with cells susceptible of being cancerous, and
correlating the presence or absence of the vimentin to the presence or absence of the cancer in the subject,
wherein the presence of the vimentin is correlated to the presence of the cancer, and
wherein the absence of the vimentin is correlated to the absence of the cancer.

38. The method of claim 37, wherein the exposing occurs in vitro.

39. The method of claim 37, wherein the exposing further comprises a step of isolating the cells from the subject and exposing the cells to the peptoid in vitro.

40. The method of claim 37, wherein the exposing occurs in vivo.

41. The method of claim 40, wherein the exposing comprises administering the peptoid to the subject in vivo.

42. The method of claim 37, wherein the detecting occurs by a method selected from the group consisting of visualization, microscopy, dark field microscopy, spectrometry, spectroscopy, colorimetric analysis, localized surface plasmon resonance (LSPR), nuclear magnetic resonance (NMR), computed tomography (CT), positron emission tomography (PET), surface plasmon resonance, electrochemistry, immunodetection, and combinations thereof.

43. (canceled)

44. The method of claim 37, wherein the cancer is selected from the group consisting of lung cancer, non-small cell lung cancer, colon cancer, esophageal cancer, breast cancer, melanoma, prostate cancer, cervical cancer, and combinations thereof.

45. The method of claim 37, further comprising a step of implementing a treatment decision, wherein the treatment decision comprises treatment of the cancer if the presence of vimentin is detected, and wherein the treatment decision comprises monitoring the subject if the presence of vimentin is not detected.

46-48. (canceled)

49. The method of claim 32, wherein the peptoid is selected from the group consisting of 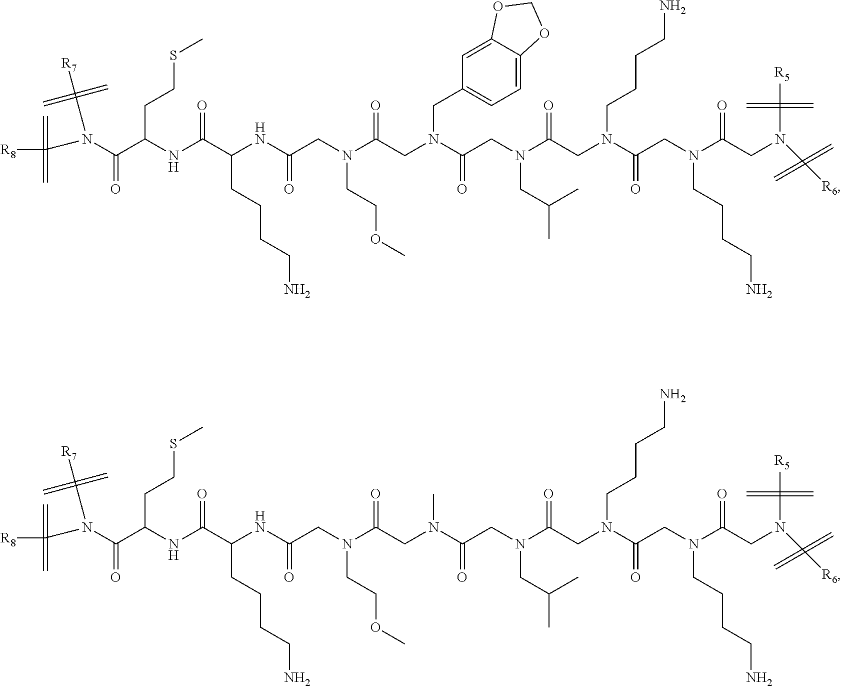 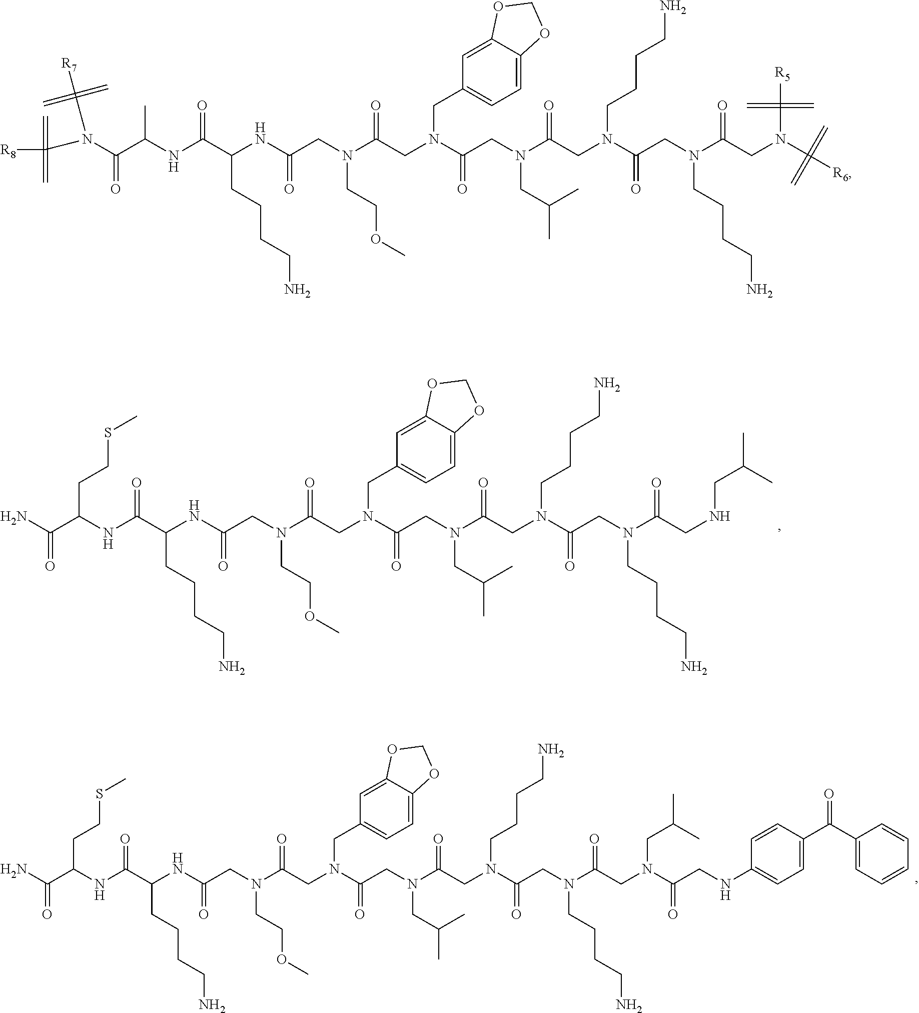 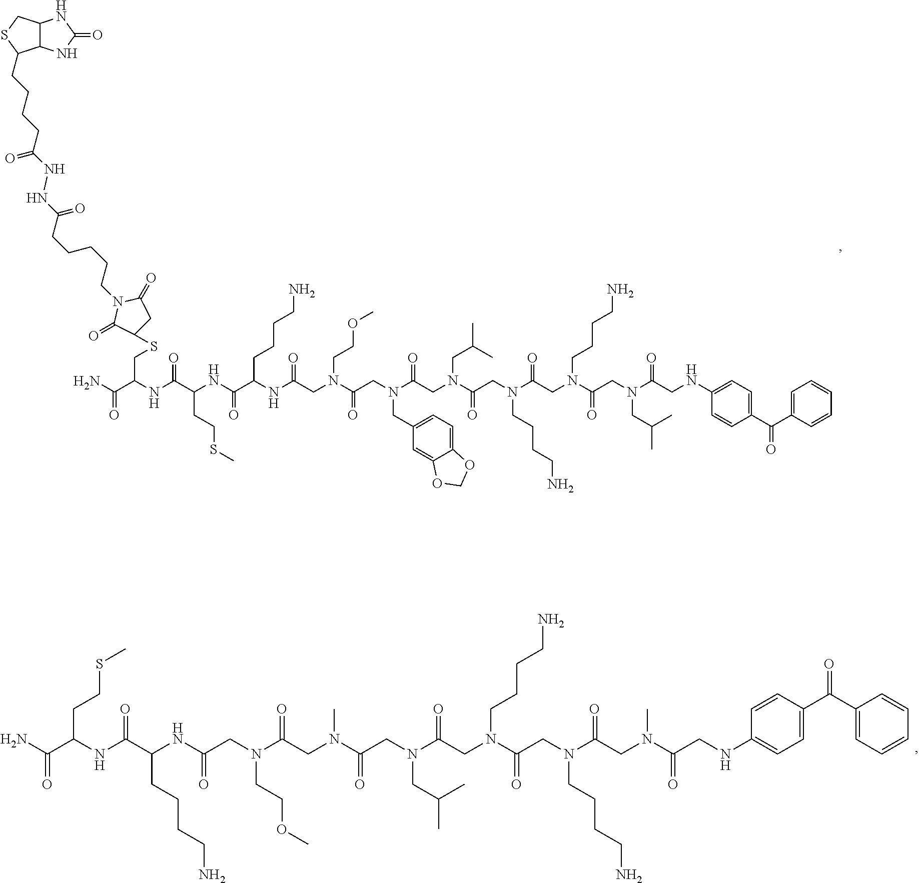 [SMILES: COCCN(CC(=O)NC(CCCCN)C(=O)NC(CCSC)C(=O)NC(CSC1CC(=O)N(CCCCCC(=O)NNC(=O)CCCCC2SCC3NC(=O)NC32)C1=O)C(N)=O)C(=O)CN(Cc1ccc2c(c1)OCO2)C(=O)CN(CC(C)C)C(=O)CN(CCCCN)C(=O)CN(CCCCN)C(=O)CN(CC(C)C)C(=O)CNc1ccc(C(=O)c2ccccc2)cc1 ; COCCN(CC(=O)NC(CCCCN)C(=O)NC(CCSC)C(N)=O)C(=O)CN(C)C(=O)CN(CC(C)C)C(=O)CN(CCCCN)C(=O)CN(CCCCN)C(=O)CN(C)C(=O)CNc1ccc(C(=O)c2ccccc2)cc1]
multimers thereof, derivatives thereof, and combinations thereof.

50. The method of claim 32, wherein the peptoid is in the form of a monomer.

51. The method of claim 32, wherein the peptoid is in the form of a multimer, wherein peptoids in the multimer are connected through one or more covalent linkages on peptoid backbones, R groups, and combinations thereof; and wherein the covalent linkages are positioned at the C-terminus of peptoids, the N-terminus of peptoids, regions proximal to the N-terminus of peptoids, middle regions of peptoids, regions proximal to the C-terminus of peptoids, and combinations thereof.

52. The method of claim 51, wherein the covalent linkages comprise the R₇ group, the R₈ group, one or more linkers, and combinations thereof.

53. The method of claim 51, wherein the multimer is selected from the group consisting of: 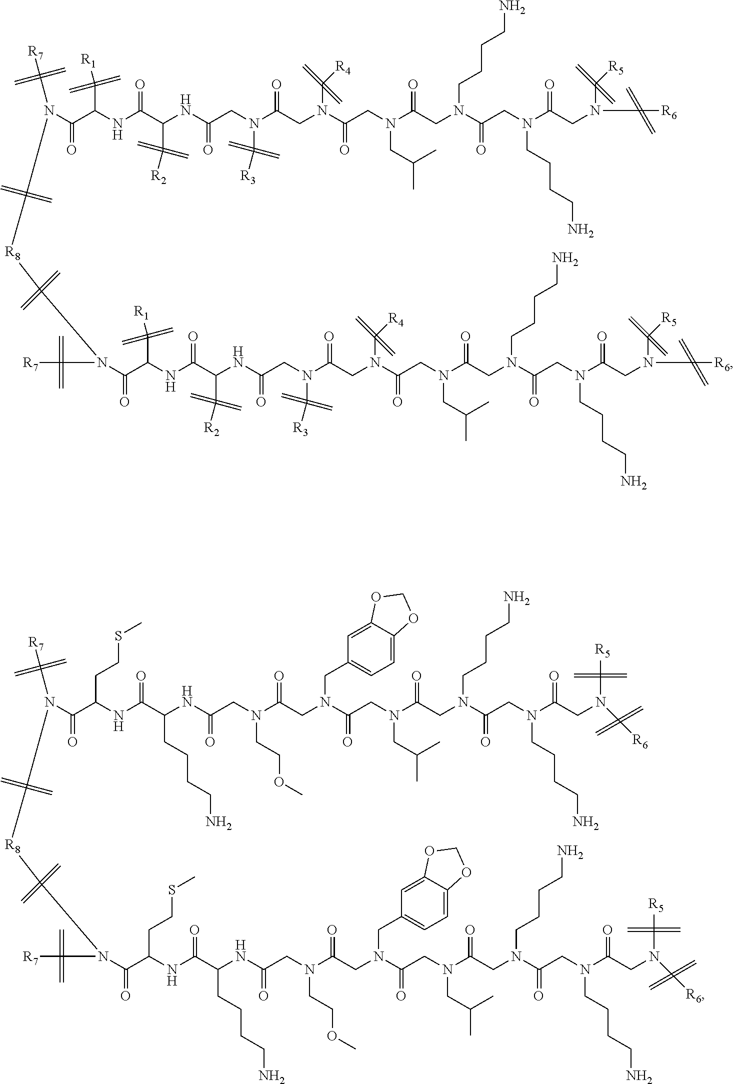 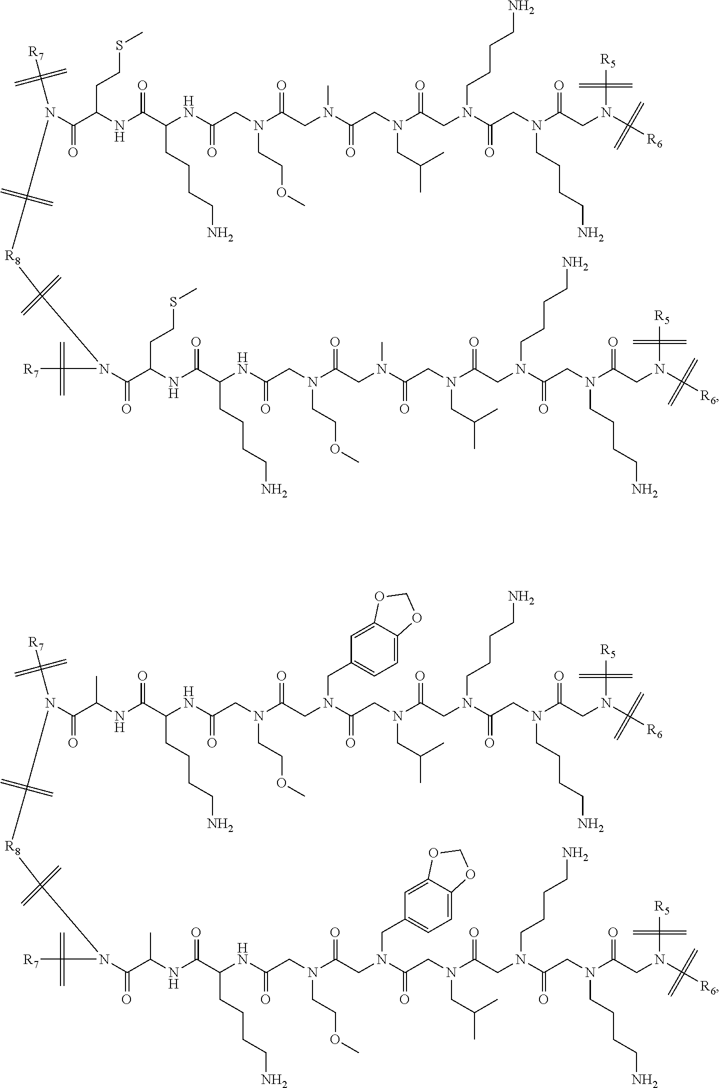
derivatives thereof, and combinations thereof.

54. The method of claim 51, wherein the peptoid is selected from the group consisting of: 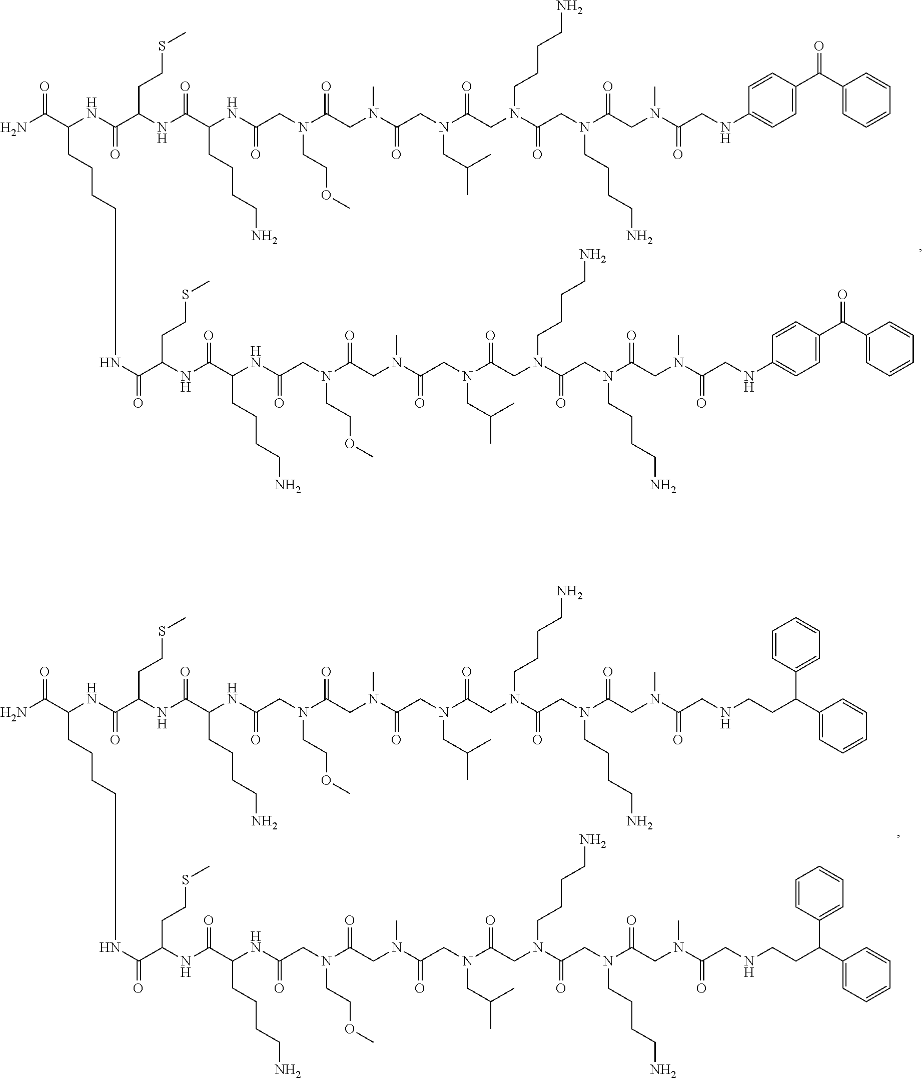 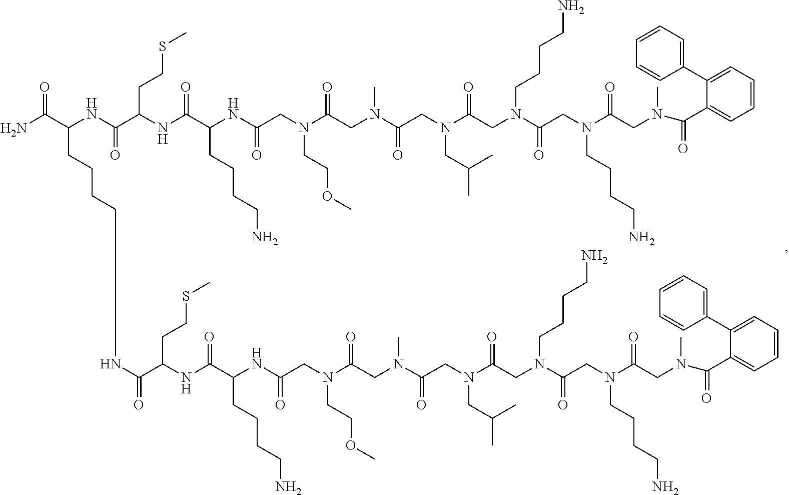 [SMILES: COCCN(CC(=O)NC(CCCCN)C(=O)NC(CCSC)C(=O)NCCCCC(NC(=O)C(CCSC)NC(=O)C(CCCCN)NC(=O)CN(CCOC)C(=O)CN(C)C(=O)CN(CC(C)C)C(=O)CN(CCCCN)C(=O)CN(CCCCN)C(=O)CN(C)C(=O)c1ccccc1-c1ccccc1)C(N)=O)C(=O)CN(C)C(=O)CN(CC(C)C)C(=O)CN(CCCCN)C(=O)CN(CCCCN)C(=O)CN(C)C(=O)c1ccccc1-c1ccccc1] 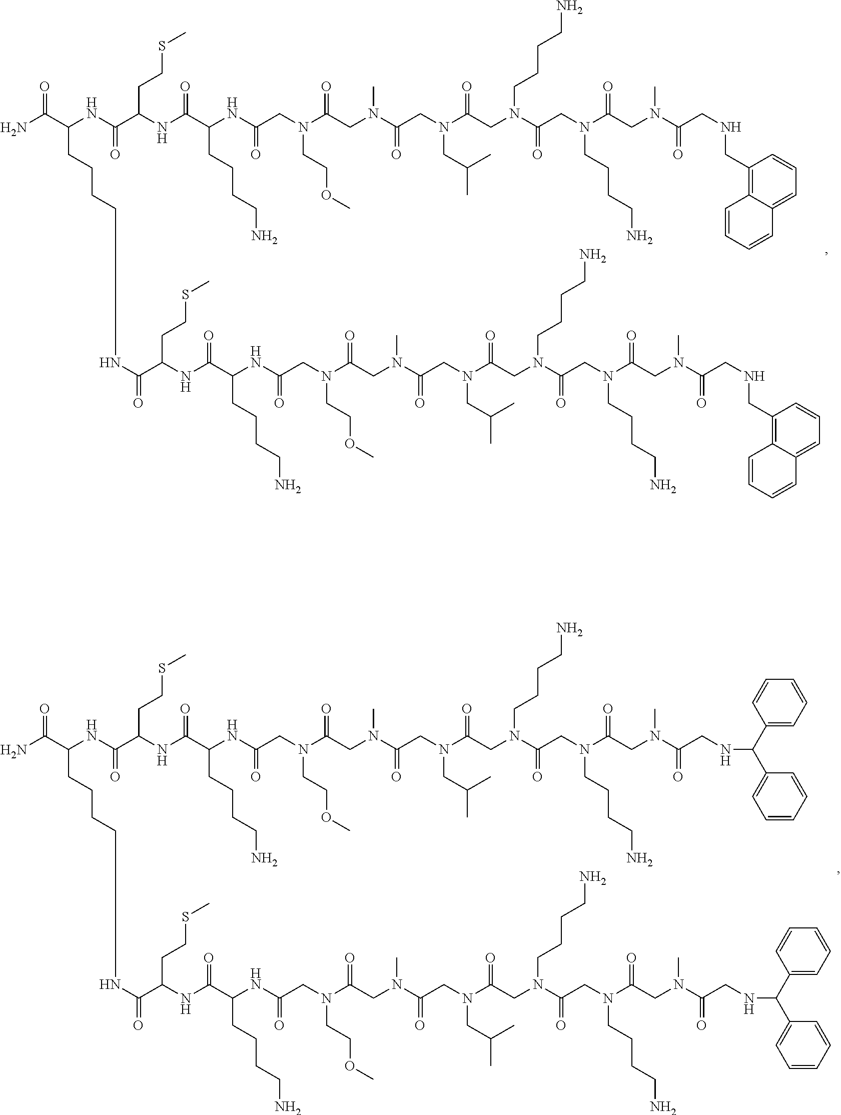 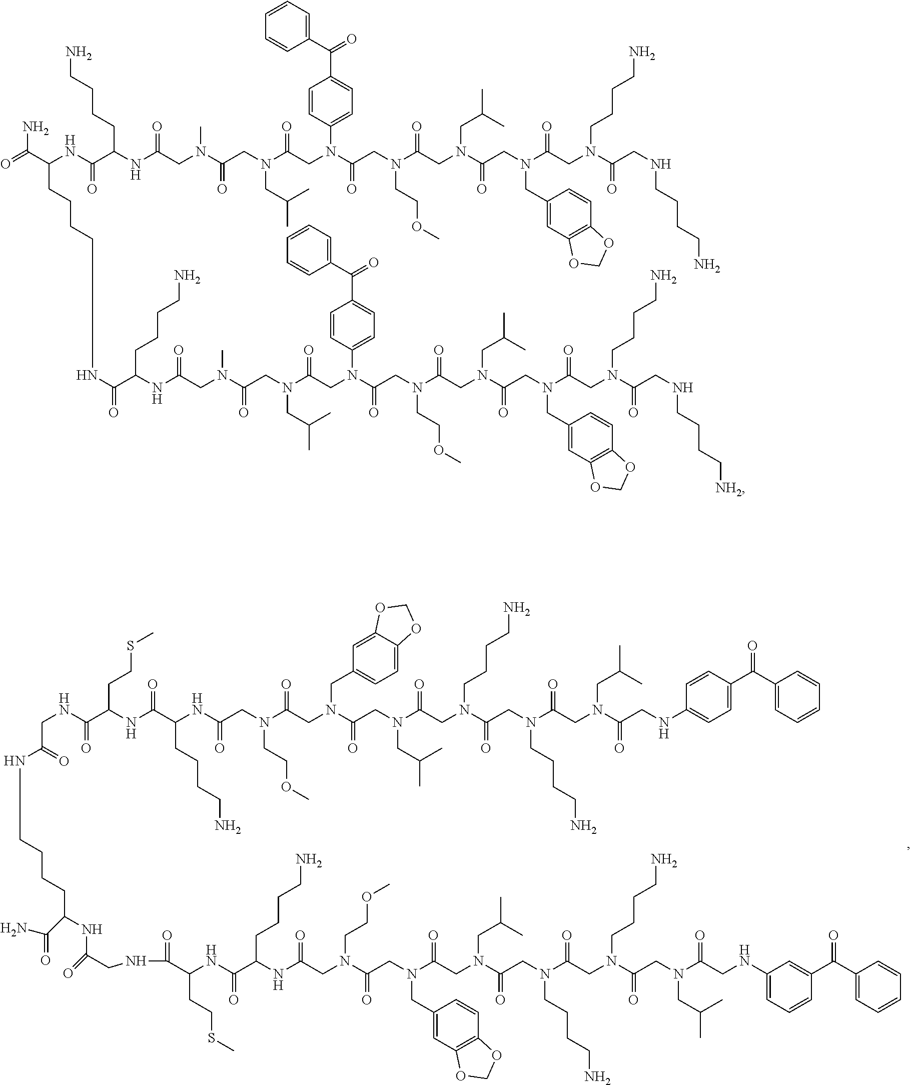 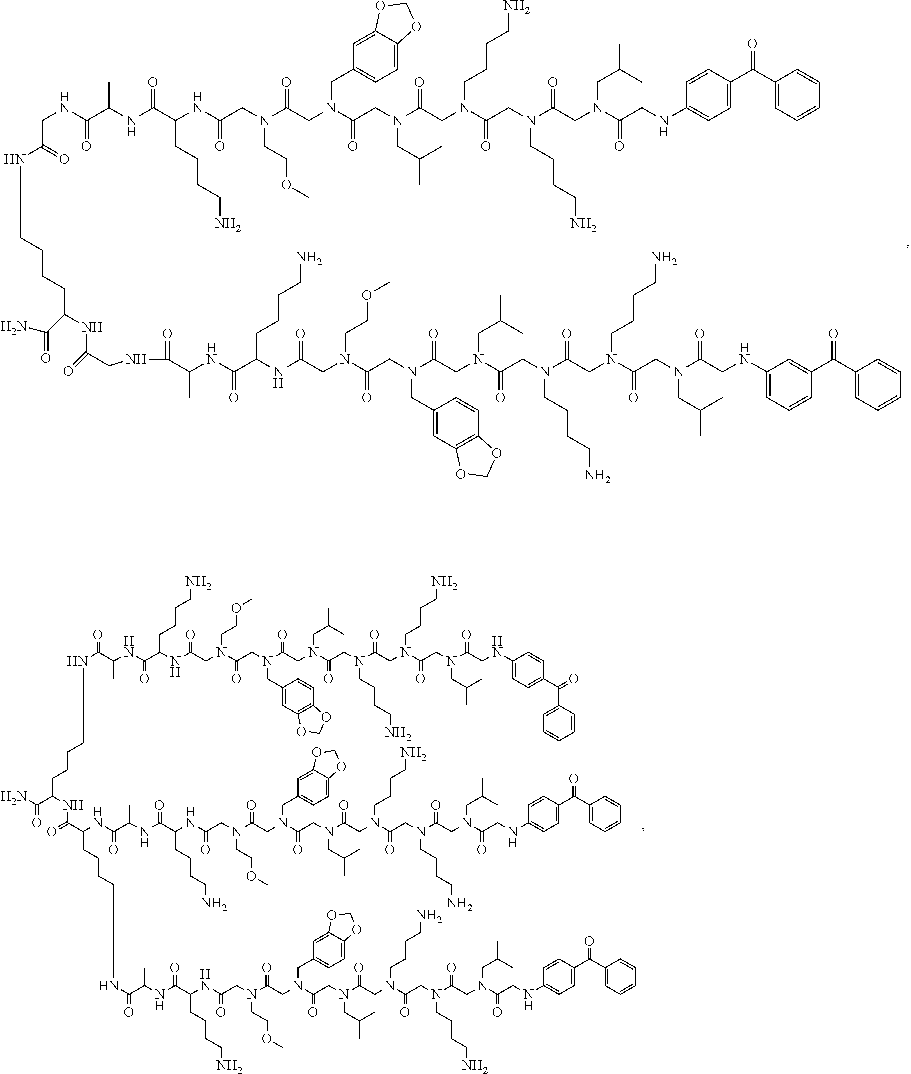 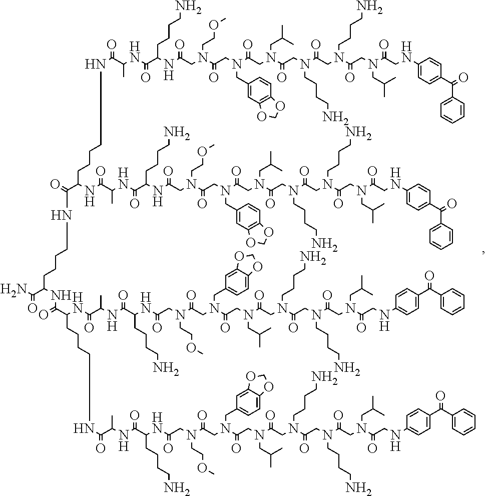
derivatives thereof, and combinations thereof.
